(19) United States
(12) Patent Application Publication
(10) Pub. No.: US 2025/0201344 A1
(43) Pub. Date: Jun. 19, 2025

(54) METHODS AND SYSTEMS FOR IDENTIFYING AN ORIGIN OF A VARIANT

(71) Applicant: GUARDANT HEALTH, INC., Palo Alto, CA (US)

(72) Inventors: Patrick C FIAUX, Brooklyn, NY (US); MingYang CAI, Palo Alto, CA (US); Shile ZHANG, San Diego, CA (US); Katie Julia QUINN, Redwood City, CA (US); Che-yu LEE, Redwood City, CA (US)

(21) Appl. No.: 18/786,211

(22) Filed: Jul. 26, 2024

Related U.S. Application Data
(60) Provisional application No. 63/516,207, filed on Jul. 28, 2023.

(51) Int. Cl. G16B 30/00; G06F 30/27; G16B 40/20

(52) U.S. Cl. CPC G16B 30/00; G06F 30/27; G16B 40/20

(57) ABSTRACT

Provided herein are methods for differentiating tumor and non-tumor (e.g., clonal hematopoiesis of indeterminate potential (CHIP)) origin nucleic acid variants from one another in a test sample obtained from a test subject at least partially using a computer. Other aspects are directed to methods of treating disease in subjects. Yet other aspects include related systems and computer readable media used to differentiating tumor and non-tumor origin nucleic acid variants from one another.

### CROSS-REFERENCE TO RELATED APPLICATIONS

This application claims the benefit of priority of U.S. Provisional Patent Application No. 63/516,207, filed Jul. 28, 2023, which is incorporated by reference herein in its entirety for all purposes.

### BACKGROUND

Liquid biopsy next generation sequencing (NGS) assays are known to observe confounding genomic signal from nucleic acid variants originating from white blood cells. Stem cells in the bone marrow ‘white blood cells’ divide to produce new blood cells, and each time a cell divides, there is a chance that a mistake in DNA replication may occur. The high rate of cell division in stem cells allow for the accumulation of mutations, producing daughter blood cells that share these mutations, even though these cells are non-cancerous. The accumulation of mutations in blood cells is called clonal hematopoiesis of indeterminate potential (CHIP). While it is well understood that variants observed in a specific subset of genes provide the majority of confounding CHIP signal, at present it is difficult to adjudicate whether the variant observed in these genes arises from white blood cell or tumor.

Accordingly, there is a need for methods of differentiating tumor and CHIP origin nucleic acid variants from one another.

### SUMMARY OF THE INVENTION

Described herein is a method comprising: determining sequence data of a plurality of sequence fragments associated with a plurality of genomic regions, wherein the sequence data includes a plurality of sequence reads, wherein the plurality of sequence reads are sequenced from the plurality of sequence fragments from a plurality of samples, wherein each sample of the plurality of samples is labeled as a tumor derived or a non-tumor derived; determining epigenetic data associated with the plurality of sequence fragments; determining, based on the sequence data and epigenetic data, a plurality of features for a predictive model; generating, based on the sequence data and epigenetic data, a predictive model according to the plurality of features. In other embodiments, determining sequence data includes obtaining a plurality of samples from a plurality of subjects, wherein the plurality of samples include a plurality of cell-free nucleic acids. In other embodiments, determining a plurality of features for a predictive model includes selecting application features from a set of candidate features. In other embodiments, selecting application features from a set of candidate features includes feature space reduction. In various embodiments, selecting application features for feature space reduction includes univariate model performance. In various embodiments, this includes for example, one or more of: a false discovery rate of smaller equal 50% (FDR<=50%) or less, and sensitivity>=1% or more, and/or cancer-specific feature exclusion as filtering criteria. In various embodiment, candidate features are derived from one or more of: variant-level information such as VAF, fragmentomics, methylation, and/or clinical test sample database, such as aggregated variant summary statistics from clinical patients, clonality, longitudinal variant variability, cancer-type variability, etc. and/or public data sets, such as COSMIC (variant frequency in cancer tissue), GnomAD (population-level allele frequencies), COSMIC mutational signatures (SBS signatures). In various embodiments, selecting application features includes excluding candidate features with greater than 50%, 60%, 70%, 80% or more correlation. In other embodiments, the plurality of features include at least one of: fragment length, a variant VAF, variant CHIP to somatic ratio, APOBEC-related cancer marker, variant measurement variability, variant maximum clonality, an age related marker, variant clonality variance, population allele frequency, ratio of methylated to unmethylated fragments, a genomic region associated with a cancer type, a genomic region associated with a methylation status, a genomic region associated with hypomethylation, or a genomic region associated with therapy response. In other embodiments, the method includes application of 1-5, 5-10, 10-15, 15-20, 20 or more features. In other embodiments, the application feature includes ratio of methylated to unmethylated fragments. In other embodiments, the fragment length is a mean length and/or length variance. In other embodiments, the fragment length is a mononucleosome and/or dinucleosome associated. In other embodiments, the features is a COSMIC related signature. In other embodiments, the age related marker is SBS88. In other embodiments, the APOBEC-related cancer marker is SBS2. In other embodiments, the variant measurement is one or more of: variability, variant maximum clonality, and variant clonality variance. In other embodiments, the epigenetic data includes at least one of: information regarding DNA methylation, histone states or modifications, inflammation-mediated cytosine damage products, or protein binding. In other embodiments, the epigenetic data associated with the plurality of sequence fragments includes determining a methylation state of the plurality of sequence fragments. In other embodiments, the methylation state of the plurality of sequence fragments includes determining at least one of: a methylation state vector or a methylated CpG density. In other embodiments, the method includes determining the methylation state vector includes: aligning the plurality of sequence reads to a reference sequence; determining, based on the aligning, a methylation status of one or more CpG sites in a sequence read of the plurality of sequence reads and a location of the one or more CpG sites; and vectorizing the methylation status of the one or more CpG sites and the locations of the one or more CpG sites to generate the methylation state vector for the sequence read of the plurality of sequence reads. In other embodiments, the methods includes determining the methylated CpG density includes: aligning the plurality of sequence reads to a reference sequence; determining, based on the aligning, a methylation status of one or more CpG sites in a sequence read of the plurality of sequence reads; determining, based on the methylation status of the one or more CpG sites in the sequence read, that the sequence read is methylated or unmethylated; determining, for the plurality of sequence reads, a count of methylated sequence reads and a count of unmethylated sequence reads; and determining, based on the count of methylated sequence reads and the count of unmethylated sequence reads, the methylated CpG density. In other embodiments, training the predictive model includes application of a machine learning algorithm. In other embodiments, the machine learning approach includes at least one of: a discriminant analysis, a decision tree, a nearest neighbor (NN) algorithm, a Bayesian network, a clustering algorithm, a neural network, a support vector machine (SVM), a logistic regression algorithm, a linear regression algorithm, a Markov model, or a principal component analysis (PCA). In other embodiments, the method includes retraining of the predictive model. In other embodiments, the method includes determining, for a subject, test sequence data comprising a plurality of sequence reads sequenced from a sample from the subject; generating test epigenetic data and/or test fragmentomic data associated with the plurality of sequence fragments; providing, to the predictive model, test sequence data, test epigenetic data, and test fragmentomic data of the subject; and determining, based on the test sequence data, the test epigenetic data, and the test fragmentomic data of the subject, an origin of at least on sequence fragment in the sequence data. In other embodiments, the method includes determining origin of at least on sequence fragment in the sequence data. In other embodiments, the origin is one of tumor derived or non-tumor derived. In other embodiments, the method includes administering one or more therapies to the subject based on the origin being tumor derived. In other embodiments, the therapies include administering chemotherapy, administering radiation therapy, or performing surgery to resect all or a portion of a tumor.

Described herein is a method comprising: obtaining sequence data comprising a plurality of sequence reads associated with a plurality of genomic regions, wherein the sequence data is generated from a sample from the subject; determining epigenetic data associated with the plurality of sequence reads; providing, to a trained predictive model, at least a portion of the sequence data and at least a portion of the epigenetic data; and determining, based on the predictive model, that the sample is tumor-derived or non-tumor derived. In other embodiments, the method includes generating the predictive model. In other embodiments, generating the predictive model includes: determining sequence data of a plurality of samples, wherein each sample of the plurality of samples is labeled as a tumor derived or a non-tumor derived; determining epigenetic data associated with the plurality of sequence reads; determining, based on a portion of the sequence data and a portion of the epigenetic data, a plurality of features for the predictive model; training, based on a portion of the sequence data and a portion of the epigenetic data, the predictive model according to the plurality of features; and outputting the predictive model based on the training. In other embodiments, the method includes testing based on a portion of the sequence data and a portion of the epigenetic data. In other embodiments, the method includes determining sequence data includes obtaining a plurality of samples from a plurality of subjects, wherein the plurality of samples include a plurality of cell-free nucleic acids. In other embodiments, determining a plurality of features for a predictive model includes selecting application features from a set of candidate features. In other embodiments, selecting application features from a set of candidate features includes feature space reduction. In various embodiments, selecting application features for feature space reduction includes univariate model performance. In various embodiments, this includes for example, one or more of: a false discovery rate of smaller equal 50% (FDR<=50%) or less, and sensitivity>=1% or more, and/or cancer-specific feature exclusion as filtering criteria. In various embodiment, candidate features are derived from one or more of: variant-level information such as VAF, fragmentomics, methylation, and/or clinical test sample database, such as aggregated variant summary statistics from clinical patients, clonality, longitudinal variant variability, cancer-type variability, etc. and/or public data sets, such as COSMIC (variant frequency in cancer tissue), GnomAD (population-level allele frequencies), COSMIC mutational signatures (SBS signatures). In various embodiments, selecting application features includes excluding candidate features with greater than 50%, 60%, 70%, 80% or more correlation. In other embodiments, the plurality of features include at least one of: fragment length, a variant VAF, variant CHIP to somatic ratio, APOBEC-related cancer marker, variant measurement variability, variant maximum clonality, an age related marker, variant clonality variance, population allele frequency, ratio of methylated to unmethylated fragments, a genomic region associated with a cancer type, a genomic region associated with a methylation status, a genomic region associated with hypomethylation, or a genomic region associated with therapy response. In other embodiments, the method includes application of 1-5, 5-10, 10-15, 15-20, 20 or more features. In other embodiments, the application feature includes ratio of methylated to unmethylated fragments. In other embodiments, the fragment length is a mean length and/or length variance. In other embodiments, the fragment length is mononucleosome and/or dinucleosome associated. In other embodiments, the features is a COSMIC related signature. In other embodiments, the age related marker is SBS88. In other embodiments, the APOBEC-related cancer marker is SBS2. In other embodiments, the variant measurement is one or more of: variability, variant maximum clonality, and variant clonality variance. In other embodiments, the plurality of genomic regions include at least one of: a genomic region known to be associated with a cancer type, a genomic region associated with a known methylation status, a genomic region known to be associated with hypomethylation, or a genomic region known to be associated with therapy response. In other embodiments, the epigenetic data includes at least one of: information regarding DNA methylation, histone states or modifications, inflammation-mediated cytosine damage products, or protein binding. In other embodiments, the method includes determining the epigenetic data includes determining a methylation state associated with the plurality of genomic regions. In other embodiments, the method includes determining the methylation state includes determining at least one of: a methylation state vector or a methylated CpG density. In other embodiments, the method includes determining the methylation state vector includes: aligning the plurality of sequence reads to a reference sequence; determining, based on the aligning, a methylation status of one or more CpG sites in a sequence read of the plurality of sequence reads and a location of the one or more CpG sites; and vectorizing the methylation status of the one or more CpG sites and the locations of the one or more CpG sites to generate the methylation state vector for the sequence read of the plurality of sequence reads. In other embodiments, the method includes determining the methylated CpG density includes: aligning the plurality of sequence reads to a reference sequence; determining, based on the aligning, a methylation status of one or more CpG sites in a sequence read of the plurality of sequence reads; determining, based on the methylation status of the one or more CpG sites in the sequence read, that the sequence read is methylated or unmethylated; determining, for the plurality of sequence reads, a count of methylated sequence reads and a count of unmethylated sequence reads; and determining, based on the count of methylated sequence reads and the count of unmethylated sequence reads, the methylated CpG density. In other embodiments, the method includes training, based on the sequence data and the epigenetic data, the predictive model according to the plurality of features includes training the predictive model according to a machine learning algorithm. In other embodiments, the machine learning approach includes at least one of: a discriminant analysis, a decision tree, a nearest neighbor (NN) algorithm, a Bayesian network, a clustering algorithm, a neural network, a support vector machine (SVM), a logistic regression algorithm, a linear regression algorithm, a Markov model, or a principal component analysis (PCA). In other embodiments, the method includes retraining the predictive model. In other embodiments, based on the sample being tumor derived, administering one or more therapies to the subject based on the origin being tumor derived. In other embodiments, therapies include administering chemotherapy, administering radiation therapy, or performing surgery to resect all or a portion of a tumor.

Described herein is a method of differentiating tumor and clonal hematopoiesis of indeterminate potential (CHIP) origin nucleic acid variants from one another in a test sample obtained from a test subject at least partially using a computer, the method comprising: obtaining sequence data comprising a plurality of sequence reads associated with a plurality of genomic regions, wherein the sequence data is generated from a sample from the subject; determining epigenetic data associated with the plurality of sequence reads; providing, to a trained predictive model, at least a portion of the sequence data and at least a portion of the epigenetic data; and determining, based on the predictive model, the presence or absence of tumor and clonal hematopoiesis of indeterminate potential (CHIP) origin nucleic acid variants in t sample. In other embodiments, the method includes generating the predictive model, wherein generating the predictive model includes: determining sequence data of a plurality of samples, wherein each sample of the plurality of samples is labeled as a tumor derived or a non-tumor derived; determining epigenetic data associated with the plurality of sequence reads; determining, based on a portion of the sequence data and a portion of the epigenetic data, a plurality of features for the predictive model; training, based on a portion of the sequence data and a portion of the epigenetic data, the predictive model according to the plurality of features; and outputting the predictive model based on the training.

Described herein is a method of treating cancer in a test subject, the method comprising:
obtaining sequence data comprising a plurality of sequence reads associated with a plurality of genomic regions, wherein the sequence data is generated from a sample from the subject; determining epigenetic data associated with the plurality of sequence reads; providing, to a trained predictive model, at least a portion of the sequence data and at least a portion of the epigenetic data; and determining, based on the predictive model, the presence or absence of tumor and clonal hematopoiesis of indeterminate potential (CHIP) origin nucleic acid variants in t sample. administering at least one therapy to the test subject based upon one or more of the differentiated tumor origin nucleic acid variants in the set of differentiated tumor and CHIP origin nucleic acid variants present in the test sample, thereby treating the cancer in the test subject.

Described herein is a method of treating cancer in a test subject, the method comprising administering at least one therapy to the test subject based upon one or more differentiated tumor origin nucleic acid variants in a set of differentiated tumor and clonal hematopoiesis of indeterminate potential (CHIP) origin nucleic acid variants present in the test sample, wherein the set of differentiated tumor and CHIP origin nucleic acid variants is produced by:
identifying, by a computer, nucleic acid variants in a set of targeted genomic regions from sequence information obtained from nucleic acids in a test sample obtained from the test subject to produce a set of identified test nucleic acid variants; identifying, by the computer, at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups; and using, by the computer, at least one trained classifier to differentiate tumor and CHIP origin nucleic acid variants from one another in the set of test nucleic acid variant-epigenetic signature groups. In other embodiments, the method includes generating the predictive model, wherein generating the predictive model includes:
determining sequence data of a plurality of samples, wherein each sample of the plurality
of samples is labeled as a tumor derived or a non-tumor derived; determining epigenetic data associated with the plurality of sequence reads; determining, based on a portion of the sequence data and a portion of the epigenetic data, a plurality of features for the predictive model; training, based on a portion of the sequence data and a portion of the epigenetic data, the predictive model according to the plurality of features; and outputting the predictive model based on the training.

In other embodiments, the epigenetic data includes differing epigenetic states or statuses exhibited by one or more epigenetic loci in a given targeted genomic region. In other embodiments, the different corresponding epigenetic signatures include differing cell-free nucleic acid (cfNA) fragment length, position, and/or endpoint density distributions. In other embodiments, a given targeted genomic region includes two or more nucleic acid variant loci. In other embodiments, the nucleic acids in the sample include cell-free nucleic acid (cfNA) fragments and/or nucleic acid molecules obtained from one or more tissues or cells in the sample. In other embodiments, the epigenetic data includes a presence or absence of methylation, hydroxymethylation, acetylation, ubiquitylation, phosphorylation, sumoylation, ribosylation, citrullination, and/or a histone post-translational modification or other histone variation for one or more of the plurality of genomic regions. In other embodiments, the method includes disregarding differentiated CHIP origin nucleic acid variants from further analysis. In other embodiments, the method includes generating at least one report that lists the tumor and CHIP origin nucleic acid variants differentiated from one another in the test sample. In other embodiments, the method includes identifying at least one cancer type associated with the differentiated tumor origin nucleic acid variants. In other embodiments, the method includes administering at least one therapy to the test subject to treat the identified cancer type. In other embodiments, the method includes administering at least one therapy to the test subject based upon one or more of the differentiated tumor origin nucleic acid variants.

Described herein is one or more non-transitory computer-readable media storing processor-executable instructions thereon that, when executed by a processor, cause the processor to perform the methods of any preceding embodiments.

Described herein is a system comprising: a computing device configured to perform the methods of any preceding claim; and an output device configured to output the predictive model.

Described herein is an apparatus, comprising: one or more processors; and memory storing processor-executable instructions that, when executed by the one or more processors, cause the apparatus to perform the methods of any preceding claim.

Described herein is one or more non-transitory computer-readable media storing processor-executable instructions thereon that, when executed by a processor, cause the processor to perform the methods of any preceding claim.

Described herein is a system comprising a computing device configured to perform the methods of any preceding claim; and an output device configured to output an indication that the sample is tumor-derived or non-tumor derived.

Described herein is an apparatus, comprising: one or more processors; and memory storing processor-executable instructions that, when executed by the one or more processors, cause the apparatus to perform the methods of any preceding claim.

Described herein is one or more non-transitory computer-readable media storing processor-executable instructions thereon that, when executed by a processor, cause the processor to perform the methods of any preceding claim.

Described herein is a system comprising: a computing device configured to perform the methods of any preceding claim; and an output device configured to output an indication that the sample is tumor-derived or non-tumor derived.

Described herein is an apparatus, comprising: one or more processors; and memory storing processor-executable instructions that, when executed by the one or more processors, cause the apparatus to perform the methods of any preceding claim.

### BRIEF DESCRIPTION OF THE FIGURES

FIG. 1. FIG. 1A. Model design. Features were engineered from Guardant internal and external public datasets and trained using 10-fold cross validation using multiple models. Only results from the Logistic Regression model are shown. Model validation was performed on an independent cohort of paired plasma and white blood cell (WBC) late-stage samples sequenced on an epigenomic panel, and healthy donors sequenced on the existing genomic panel.

FIG. 1B. demonstrates measurement of WBC sequencing. FIG. 1C. illustrates an annotation workflow. Here, one can determine the presence of CHIP (hereinafter also known as (“CH”) variants with a classification process including determination that WBC VAF>plasma VAF as a CH variant, if plasma VAF>WBC VAF*10 as somatic variant, or if plasma VAF>WBC VAF, utilization of a Fisher's exact test to rule out presence of variant in WBC from contamination from plasma. One could also impose additional filtering criteria needed to determine probability of detection in WBC based on coverage when no WBC variant is detected.

FIG. 2. Model performance. Predictions for tumor and non-tumor status were compared to WBC confirmation in 713 somatic SNV/Indels from 72 paired plasma and WBC epigenomic detection assay samples and for validation 243 somatic SNV/Indels from 76 paired plasma and healthy donors on genomic detection assay. Lower confirmation rate in WBC sequencing observed for low VAF variants (<0.6%) likely attributed to the limit of detection in for WBC variant calling and/or possible non-WBC lineage origin.

FIG. 3. Feature importance and examples. FIG. 3A. Top 10 features ranked by relative importance on validation dataset. Individual gene names were included with one-hot encoding. FIG. 3B. Highly ranked engineered features include clonality, defined as VAF/tumor fraction as measured by methylation or max somatic VAF (left), VAF variation across timepoints (middle), mean percentage (right) and FIG. 3C. uniformity in variant prevalence across solid tumor cancer types in the Guardant plasma database. FIG. 3D. Additional exemplary features and their application is shown.

FIG. 4. Number of variants within each gene predicted as non-tumor or tumor-derived as confirmed by WBC or model prediction in the late-stage validation cohort. Variant counts are shown for the genes with cfDNA variants most commonly confirmed in WBC samples, along with counts in clinically actionable gene (BRCA1, BRAF, KRAS, ESR1, ATM, CHEK2). Most frequent WBC-confirmed genes are consistent with previous reports, including high prevalence of clonal hematopoiesis in ATM and CHEK2 (*).

FIG. 5. Proportion of variants detected in WBC or predicted as non-tumor in late-stage validation cohort by age ranges. It has been reported that variants predicted or confirmed as non-tumor are highly correlated with age.

FIG. 6. is a flowchart illustrating an example training method.

FIG. 7. is an illustration of an exemplary process flow for using a machine learning-based classifier.

FIG. 8. shows an example method.

FIG. 9. shows an example method.

FIG. 10. shows an example method.

FIG. 11. shows an example method.

FIG. 12. shows an example method.

FIG. 13. Feature selection and model building process. A correlation check prevents correlation from exceeding a threshold (e.g., 70%). Feature space reduction included univariate model performance. Here, a false discovery rate of smaller equal 50% (FDR<=50%) and sensitivity>=1% filtering criteria applied, along with cancer-specific feature exclusion to remove bias in training.

FIG. 14. Buffy-plasma data: Clinically relevant genes from NHC. Example of ground truth state determined by matched buffy.

FIG. 15. FIG. 15A. CHIP classifier features: Exemplary model uses 14 features and shows best performance in both validation and testing data. Exemplary sample level features depicted. FIG. 15B. Performance receiver operator characteristics (ROC), including multi-feature model, exemplary conventional model, using rules tables for including/excluding, without multi-feature generation and reduced feature model obtained by CH variant labeling based on the presence of the same SNV/indel in paired cfDNA and buffy coat.

FIG. 16. CHIP classifier features: All features capture differences between CHIP and Somatic variants.

FIG. 17. Performance on clinically relevant genes: buffy-plasma matched samples, performance for multi-feature model, exemplary conventional model using rules tables for including/excluding, without multi-feature generation and reduced feature model.

FIG. 18. Performance on clinically relevant genes: buffy-plasma validation data depicting gene-level performance. No high FP genes detected in current classifier.

FIG. 19. Performance on clinically relevant genes: buffy-plasma matched samples depicting Validation data, VAF bins. No high FP bins detected in current classifier.

FIG. 20. Performance on clinically relevant genes: buffy-plasma matched samples validation data. All recorded cancer types. No high FP cancer types detected in current classifier.

FIG. 21. Performance on clinically relevant genes: tissue-plasma matched samples.

FIG. 22. Performance on clinically relevant genes: tissue-plasma matched samples. Specificity evaluation in low-VAF bins. No high FP bins below 0.5% detected in current classifier.

FIG. 23. Buffy-plasma data: Clinically relevant genes from exemplary panel of over 700 genes. Example of ground truth state determined by matched buffy.

FIG. 24. Performance on panel-wide variants: buffy-plasma matched samples performance for multi-feature model, exemplary conventional model using rules tables for including/excluding, without multi-feature generation and reduced feature model.

FIG. 25. Performance on panel-wide variants tissue-plasma matched samples.

FIG. 26. Actionable variant performance: buffy-plasma matched samples. Current classifier achieved 100% accuracy and 0% FRP.

FIG. 27. Actionable variant performance: tissue-plasma matched samples. Current classifier achieved 0% FRP.

FIG. 28. Actionable variant classification indicates very low FPR, exemplary panel including approximately 80 genes.

FIG. 29. Actionable variant performance, plasma ctDNA-. Most CHIP calls in known KRAS CHIP variant K117N.

FIG. 30. Summary of current model performance.

FIG. 31. CHIP classifier algorithm overview.

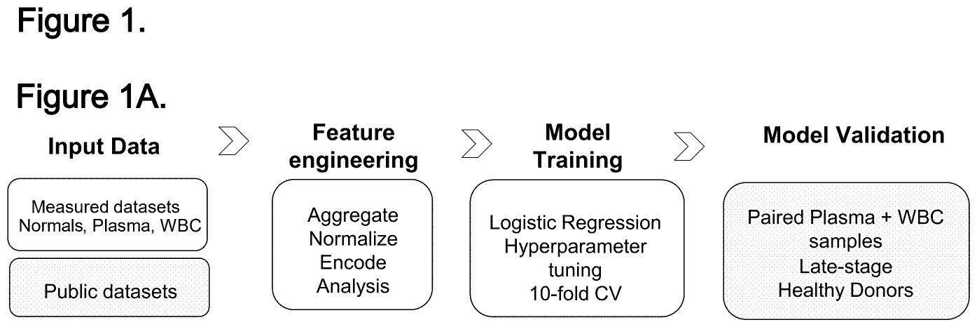

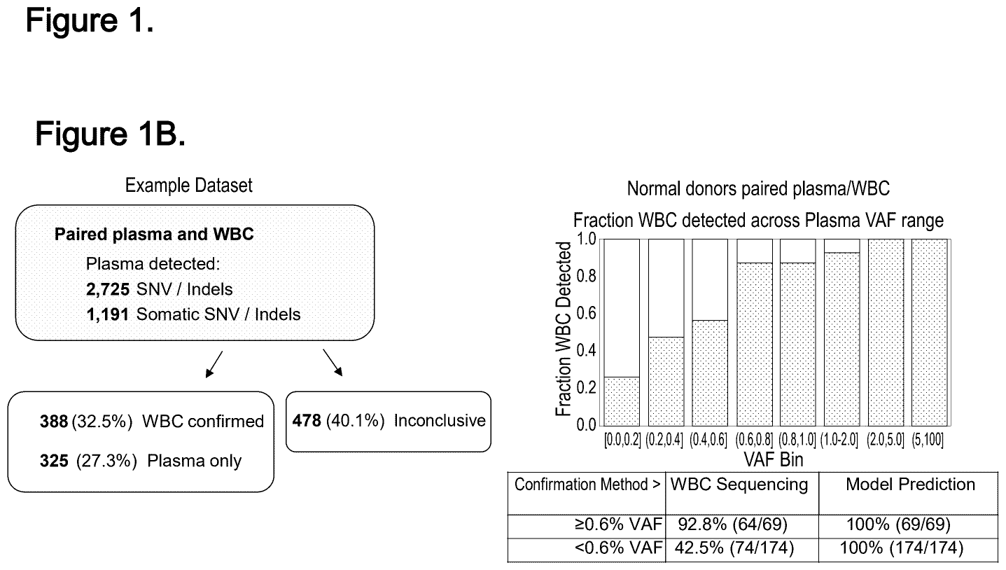

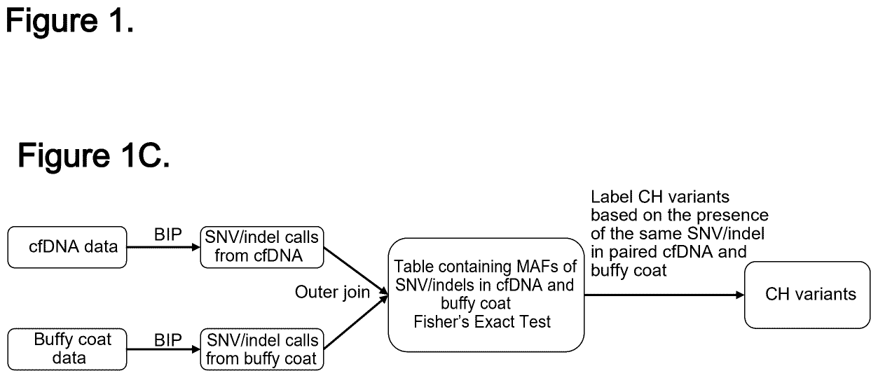

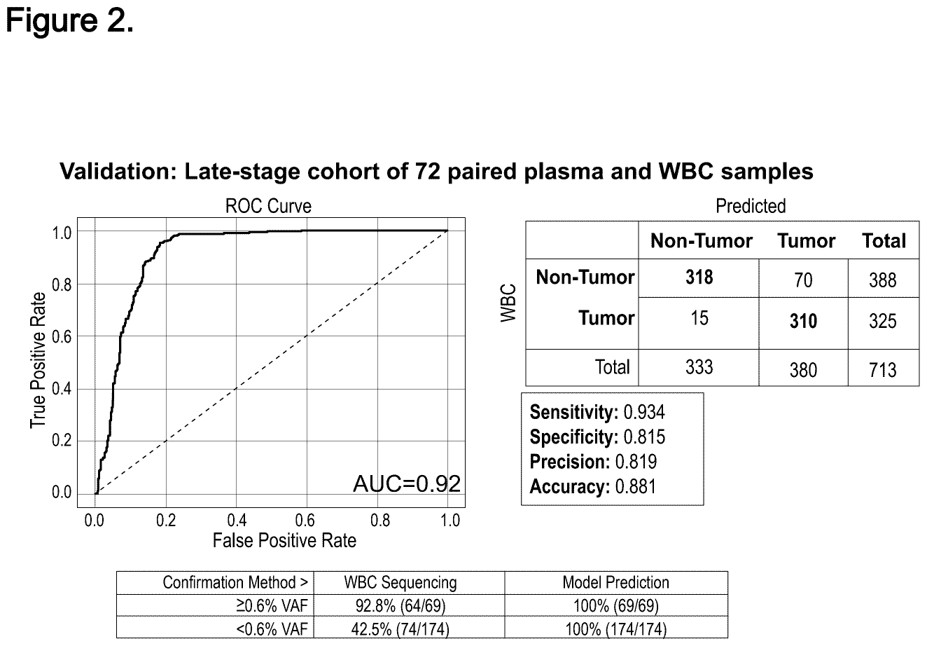

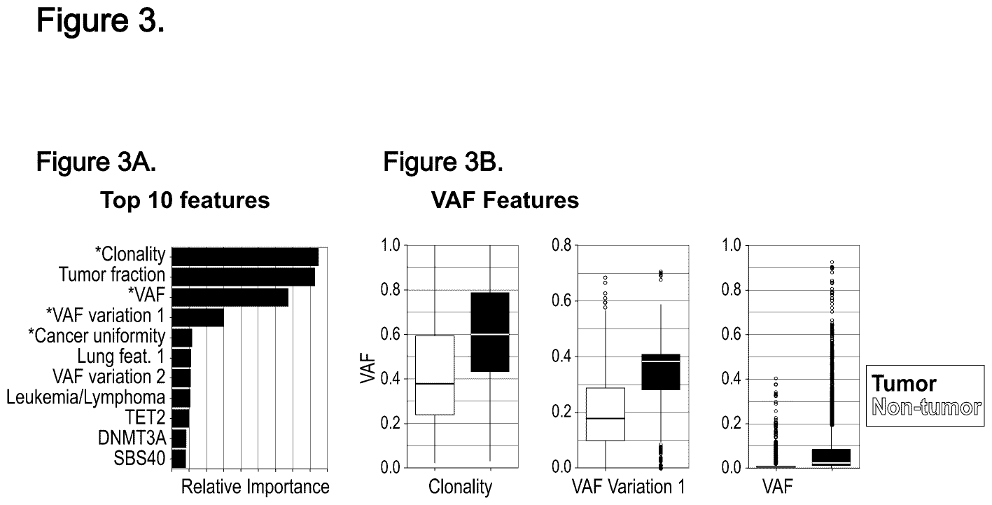

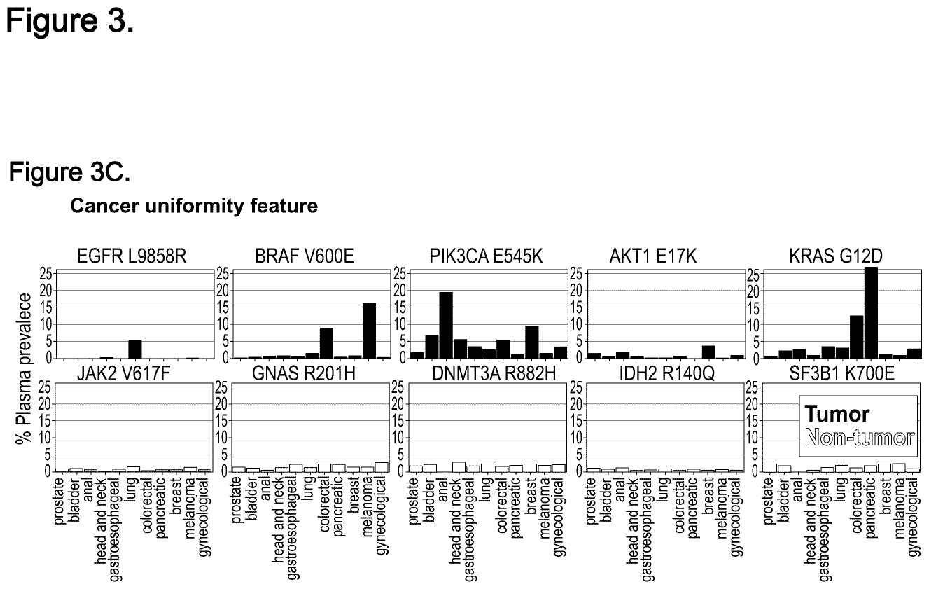

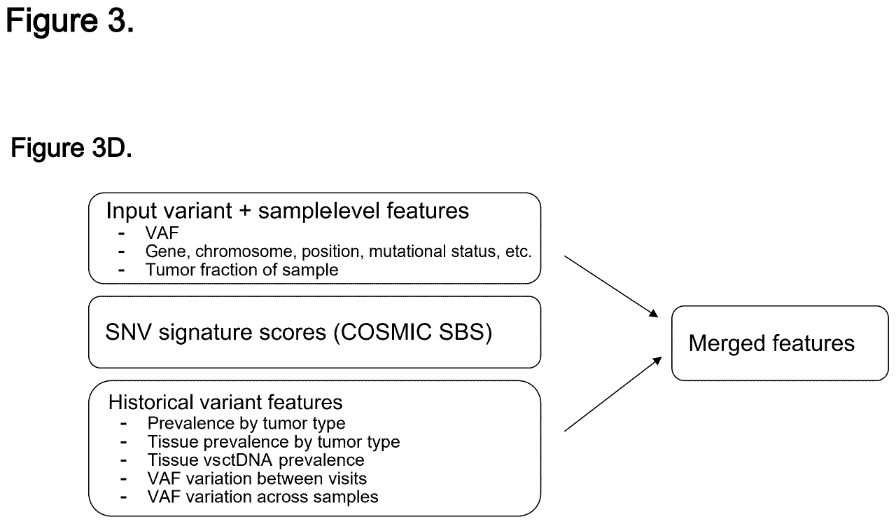

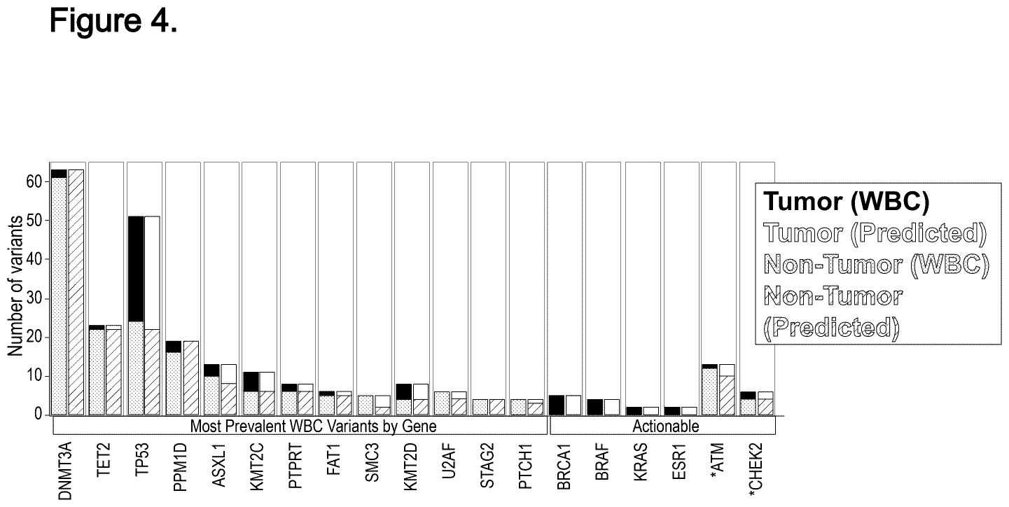

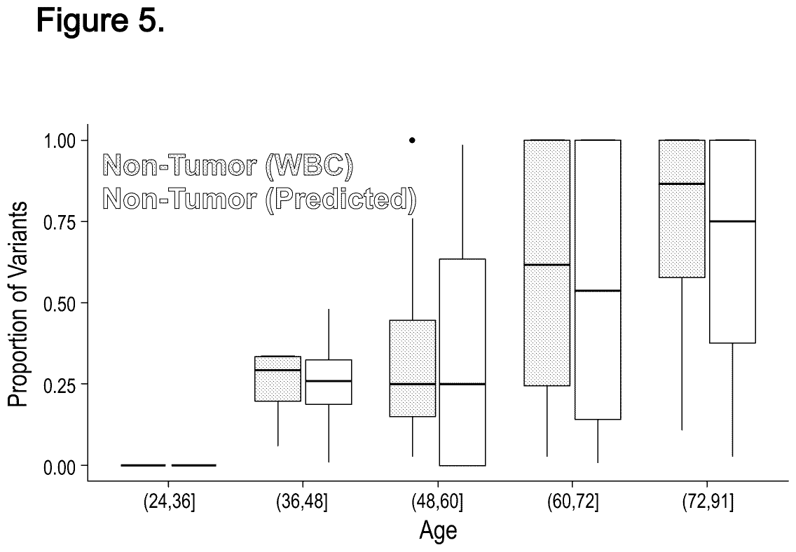

### DETAILED DESCRIPTION

While various embodiments of the disclosure have been shown and described herein, those skilled in the art will understand that such embodiments are provided by way of example only. Numerous variations, changes, and substitutions may occur to those skilled in the art without departing from the disclosure. It should be understood that various alternatives to the embodiments of the disclosure described herein may be employed.

The term “about” and its grammatical equivalents in relation to a reference numerical value can include a range of values up to plus or minus 10% from that value. For example, the amount “about 10” can include amounts from 9 to 11. The term “about” in relation to a reference numerical value can include a range of values plus or minus 10%, 9%, 8%, 7%, 6%, 5%, 4%, 3%, 2%, or 1% from that value.

The term “at least” and its grammatical equivalents in relation to a reference numerical value can include the reference numerical value and greater than that value. For example, the amount “at least 10” can include the value 10 and any numerical value above 10, such as 11, 100, and 1,000.

The term “at most” and its grammatical equivalents in relation to a reference numerical value can include the reference numerical value and less than that value. For example, the amount “at most 10” can include the value 10 and any numerical value under 10, such as 9, 8, 5, 1, 0.5, and 0.1.

As used herein the singular forms “a”, “an”, and “the” can include plural referents unless the context clearly dictates otherwise. Thus, for example, reference to “a cell” can include a plurality of such cells and reference to “the culture” can include reference to one or more cultures and equivalents thereof known to those skilled in the art, and so forth. All technical and scientific terms used herein can have the same meaning as commonly understood to one of ordinary skill in the art to which this disclosure belongs unless clearly indicated otherwise.

Cancer can be indicated by epigenetic variations, such as methylation. Examples of methylation changes in cancer include local gains of DNA methylation in the CpG islands at the transcription start site (TSS) of genes involved in normal growth control, DNA repair, cell cycle regulation, and/or cell differentiation. This hypermethylation can be associated with an aberrant loss of transcriptional capacity of involved genes and occurs at least as frequently as point mutations and deletions as a cause of altered gene expression. DNA methylation profiling can be used to detect regions with different extents of methylation (“differentially methylated regions” or “DMRs”) of the genome that are altered during development or that are perturbed by disease, for example, cancer or any cancer-associated disease. The genome of cancer cells harbor imbalance in the above DNA methylation patterns, and therefore in functional packaging of the DNA. The abnormalities of chromatin organization are therefore coupled with methylation changes and may contribute to enhanced cancer profiling when analyzed jointly. Combining MBD-partitioning with fragmentomic data, such as fragment mapped starts and stops positions (correlated with nucleosome positions), fragment length and associated nucleosome occupancy, can be used for chromatin structure analysis in hypermethylation studies with the aim to improve biomarker detection rate.

Methylation profiling can involve determining methylation patterns across different regions of the genome. For example, after partitioning molecules based on extent of methylation (e.g., relative number of methylated sites per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

A characteristic of nucleic acid molecules may be a modification, which may include various chemical or protein modifications (i.e. epigenetic modifications). Non-limiting examples of chemical modification may include, but are not limited to, covalent DNA modifications, including DNA methylation. In some embodiments, DNA methylation includes addition of a methyl group to a cytosine at a CpG site (a cytosine followed by a guanine in a nucleic acid sequence). In some embodiments, DNA methylation includes addition of a methyl group to adenine, such as in N6-methyladenine. In some embodiments, DNA methylation is 5-methylation (modification of the 5th carbon of the 6 carbon ring of cytosine). In some embodiments, 5-methylation includes addition of a methyl group to the 5C position of the cytosine to create 5-methylcytosine (m5c). In some embodiments, methylation includes a derivative of m5c. Derivatives of m5c include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6 carbon ring of cytosine). In some embodiments, 3C methylation includes addition of a methyl group to the 3C position of the cytosine to generate 3-methylcytosine (3mC). Other examples include N6-methyladenine or glycosylation. DNA methylation includes addition of methyl groups to DNA (e.g. CpG) and can change the expression of methylated DNA region. Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

A CpG dyad is the dinucleotide CpG (cytosine-phosphate-guanine, i.e. a cytosine followed by a guanine in a 5′→3′ direction of the nucleic acid sequence) on the sense strand and its complementary CpG on the antisense strand of a double-stranded DNA molecule. CpG dyads can be either fully methylated or hemi-methylated (methylated on one strand only).

The CpG dinucleotide is underrepresented in the normal human genome, with the majority of CpG dinucleotide sequences being transcriptionally inert (e.g. DNA heterochromatic regions in pericentromeric parts of the chromosome and in repeat elements) and methylated. However, many CpG islands are protected from such methylation especially around transcription start sites (TSS).

Specifically, in accordance with methods and techniques described herein, epigenomic measurement of tumor fraction can improve negative predicition. Assumig some tumor fraction based on the data and provide the likelihood of the absence of a variant>30% clonality, inflation of tumor fraction prediction (CNV on epi-MAF gene, CHIP, ect) can cause overconfidence in negative prediction, while deflation of tumor-fraction (TND, ect) leads to the inability to make confident negative calls. Static parameters include s0ub-clonal variant purity boundary (30%), prior variant likelihood, and mutual exclusivity or co-occurrence with other variants. Key derived parameters include tumor fraction and cancer tissue of origin. Taking into account these parameters, a probability distribution of tumor fraction based on methylation data can be measured.

Protein modifications include binding to components of chromatin, particularly histones including modified forms thereof, and binding to other proteins, such as proteins involved in replication or transcription. The disclosure provides methods of processing and analyzing nucleic acids with different extents of modification, such that the nature of their original modification is correlated with a nucleic acid tag and can be decoded by sequencing the tag when nucleic acids are analyzed. Genetic variation of sample nucleic acid modifications can then be associated with the extent of modification (epigenetic variation) of that nucleic acid in the original sample, include single stranded (e.g., ssDNA or RNA) or double stranded molecules (e.g., dsDNA).

The loss of DNA can reduce the presence of one or more types of DNA such that the presence of the one or more types of DNA such as cfDNA, is difficult to detect. In one or more additional scenarios, existing methods to measure DNA methylation, such as enrichment or depletion methods, can have a relatively high level of resolution, such as about 100 base pairs (bp) to about 200 bp that can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determinations of tumor fraction estimates can impact diagnosis and/or treatment decisions for individuals.

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed of being susceptible to cancer or any cancer-associated genetic mutations/disorders.

The volume of plasma can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample includes an epigenetic variant associated with the presence of a genetic variant, wherein the sample does not comprise the genetic variant.

Exemplary amounts of cell-free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, IRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Analytes

Analytes can include nucleic acid analytes, and non-nucleic acid analytes. The disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In general, the systems, apparatus, methods, and compositions can be used to analyze any number of analytes, further including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

One or more nucleic acid analytes and/or non-nucleic acid analytes constitute a set of molecular interactions in a biological system under study (e.g., cells), which may be regarded as “interactome”—the molecular interactions that occur between molecules belonging to different biochemical families (proteins, nucleic acids, lipids, carbohydrates, etc.) and also within a given family. In various embodiments, an interactome is a protein-DNA interactome (network formed by transcription factors (and DNA or chromatin regulatory proteins) and their target genes. In other embodiments, interactome refers to protein-protein interaction network (PPI), or protein interaction network (PIN). The methods described herein allow for study and analysis of the interactome. Techniques such as proteogenomics (whole genome sequencing, whole exome sequencing and RNA-seq, and mass spectrometry as examples) can support study of the interactome.

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens is changing during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and mutation analyses alone or in combination.

The present methods can be used to diagnose, prognose, monitor or observe cancers. or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Determination of 5-Methylcytosine Pattern of Nucleic Acids

Bisulfite-based sequencing and variants thereof provides a means of determining the methylation pattern of a nucleic acid. In some embodiments, determining the methylation pattern includes distinguishing 5-methylcytosine (5mC) from non-methylated cytosine. In some embodiments, determining methylation pattern includes distinguishing N6-methyladenine from non-methylated adenine. In some embodiments, determining the methylation pattern includes distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxylmethylcystosine) are not converted. Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid including a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51 (15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methyl-ated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this should is not limiting. Variants of BIS-seq have been developed to identify hydroxymethylated cytosines (5hmC; OX-BS-seq, TAB-seq), formylcytosine (5fC; redBS-seq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Partitioning the Sample into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein), and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher level of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled, and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

Subjecting the First Subsample to a Procedure that Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-BGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Enriching/Capturing Step, Amplification, Adaptors, Barcodes

In some embodiments, methods disclosed herein comprise a step of capturing one or more sets of target regions of DNA, such as cfDNA. Capture may be performed using any suitable approach known in the art. In some embodiments, capturing includes contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally including the first nucleobase (e.g., hmC) from DNA not originally including the first nucleobase, such as hmC-seal), capturing may be performed on any, any two, or all of the DNA originally including the first nucleobase (e.g., hmC), the DNA not originally including the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein includes capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein includes contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test fsor perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions including captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Captured Set

In some embodiments, a captured set of DNA (e.g., cfDNA) is provided. With respect to the disclosed methods, the captured set of DNA may be provided, e.g., by performing a capturing step after a partitioning step as described herein. The captured set may comprise DNA corresponding to a sequence-variable target region set, an epigenetic target region set, or a combination thereof. In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, including, respectively, DNA corresponding to a sequence-variable target region set and DNA corresponding to an epigenetic target region set. The first and second captured sets may be combined to provide a combined captured set.

In some embodiments in which a captured set including DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, a 90- to 100-fold greater concentration, a 10- to 20-fold greater concentration, a 10- to 40-fold greater concentration, a 10- to 50-fold greater concentration, a 10- to 70-fold greater concentration, or a 10- to 100-fold greater concentration. The degree of difference in concentrations accounts for normalization for the footprint sizes of the target regions, as discussed in the definition section.

### Epigenetic Target Region Set

The epigenetic target region set may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein. In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### Hypermethylation Variable Target Regions

In some embodiments, the epigenetic target region set includes one or more hypermethylation variable target regions. In general, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In an example, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions include one or more genomic regions, where the cfDNA molecules in those regions do not differ in methylation state in cancer subjects relative to cfDNA from healthy subjects, but the presence/increased quantity of hypermethylated cfDNA in those regions is indicative of a particular tissue type (e.g., cancer origin) and is presented as cfDNA with increased apoptosis (e.g. tumor shedding) into circulation.

Hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

Global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells. In an example, hypomethylation variable target regions can include regions that do not necessarily differ in methylation state in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation variable target regions. In some embodiments, hypomethylation variable target regions include one or more genomic regions, where the cfDNA molecules in those regions do not differ in methylation state in cancer subjects relative to cfDNA from healthy subjects, but the presence/increased quantity of hypomethylated cfDNA in those regions is indicative of a particular tissue type (e.g., cancer origin) and is presented as cfDNA with increased apoptosis (e.g. tumor shedding) into circulation.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation variable target regions overlap or comprise one or both of these regions.

In some embodiments, the probes for the epigenetic target region set comprise probes specific for one or more hypomethylation variable target regions. The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or including nucleotides 8403565-8953708 and/or 151104701-151106035 of human chromosome

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. Subjects

In some embodiments, the DNA (e.g., cfDNA) is obtained from a subject having a cancer. In some embodiments, the DNA (e.g., cfDNA) is obtained from a subject suspected of having a cancer. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Compositions Including Captured DNA

Provided herein is a combination including first and second populations of captured DNA. The first population may comprise or be derived from DNA with a cytosine modification in a greater proportion than the second population. The first population may comprise a form of a first nucleobase originally present in the DNA with altered base pairing specificity and a second nucleobase without altered base pairing specificity, wherein the form of the first nucleobase originally present in the DNA prior to alteration of base pairing specificity is a modified or unmodified nucleobase, the second nucleobase is a modified or unmodified nucleobase different from the first nucleobase, and the form of the first nucleobase originally present in the DNA prior to alteration of base pairing specificity and the second nucleobase have the same base pairing specificity. The second population does not comprise the form of the first nucleobase originally present in the DNA with altered base pairing specificity. In some embodiments, the cytosine modification is cytosine methylation. In some embodiments, the first nucleobase is a modified or unmodified cytosine and the second nucleobase is a modified or unmodified cytosine. The first and second nucleobase may be any of those discussed herein in the Summary or with respect to subjecting the first subsample to a procedure that affects a first nucleobase in the DNA differently from a second nucleobase in the DNA of the first subsample.

In some embodiments, the first population includes a sequence tag selected from a first set of one or more sequence tags and the second population includes a sequence tag selected from a second set of one or more sequence tags, and the second set of sequence tags is different from the first set of sequence tags. The sequence tags may comprise barcodes.

In some embodiments, the first population includes protected hmC, such as glucosylated hmC. In some embodiments, the first population was subjected to any of the conversion procedures discussed herein, such as bisulfite conversion, Ox-BS conversion, TAB conversion, ACE conversion, TAP conversion, TAPSB conversion, or CAP conversion. In some embodiments, the first population was subjected to protection of hmC followed by deamination of mC and/or C. In some embodiments of the combination, the first population includes or was derived from DNA with a cytosine modification in a greater proportion than the second population and the first population includes first and second subpopulations, and the first nucleobase is a modified or unmodified nucleobase, the second nucleobase is a modified or unmodified nucleobase different from the first nucleobase, and the first nucleobase and the second nucleobase have the same base pairing specificity. In some embodiments, the second population does not comprise the first nucleobase. In some embodiments, the first nucleobase is a modified or unmodified cytosine, and the second nucleobase is a modified or unmodified cytosine, optionally wherein the modified cytosine is mC or hmC. In some embodiments, the first nucleobase is a modified or unmodified adenine, and the second nucleobase is a modified or unmodified adenine, optionally wherein the modified adenine is mA.

In some embodiments, the first nucleobase (e.g., a modified cytosine) is biotinylated. In some embodiments, the first nucleobase (e.g., a modified cytosine) is a product of a Huisgen cycloaddition to β-6-azide-glucosyl-5-hydroxymethylcytosine that includes an affinity label (e.g., biotin).

In any of the combinations described herein, the captured DNA may comprise cfDNA. The captured DNA may have any of the features described herein concerning captured sets, including, e.g., a greater concentration of the DNA corresponding to the sequence-variable target region set (normalized for footprint size as discussed above) than of the DNA corresponding to the epigenetic target region set. In some embodiments, the DNA of the captured set includes sequence tags, which may be added to the DNA as described herein. In general, the inclusion of sequence tags results in the DNA molecules differing from their naturally occurring, untagged form.

The combination may further comprise a probe set described herein or sequencing primers, each of which may differ from naturally occurring nucleic acid molecules. For example, a probe set described herein may comprise a capture moiety, and sequencing primers may comprise a non-naturally occurring label.

### Computer Systems, Processing of Real World Evidence (RWE)

Methods of the present disclosure can be implemented using, or with the aid of, computer systems. For example, such methods may comprise: partitioning the sample into a plurality of subsamples, including a first subsample and a second subsample, wherein the first subsample includes DNA with a cytosine modification in a greater proportion than the second subsample; subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample.

In an aspect, the present disclosure provides a non-transitory computer-readable medium including computer-executable instructions which, when executed by at least one electronic processor, perform at least a portion of a method including: collecting cfDNA from a test subject; capturing a plurality of sets of target regions from the cfDNA, wherein the plurality of target region sets includes a sequence-variable target region set and an epigenetic target region set, whereby a captured set of cfDNA molecules is produced; sequencing the captured cfDNA molecules, wherein the captured cfDNA molecules of the sequence-variable target region set are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the likelihood that the subject has cancer.

The code can be pre-compiled and configured for use with a machine have a processer adapted to execute the code or can be compiled during runtime. The code can be supplied in a programming language that can be selected to enable the code to execute in a pre-compiled or as-compiled fashion.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

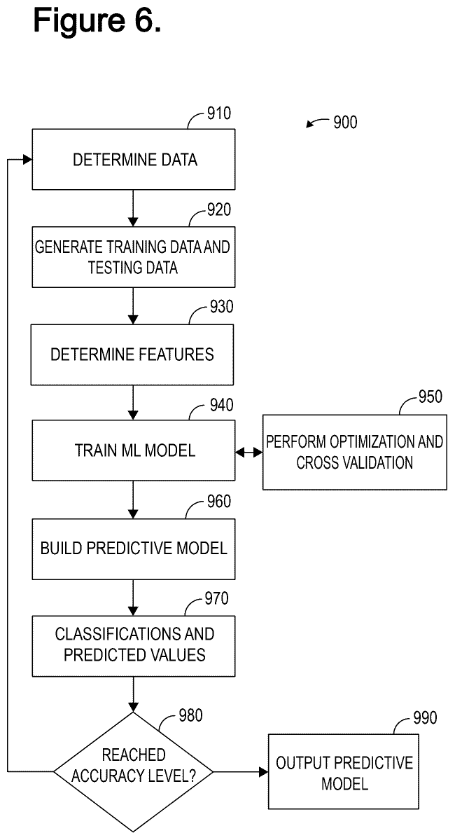

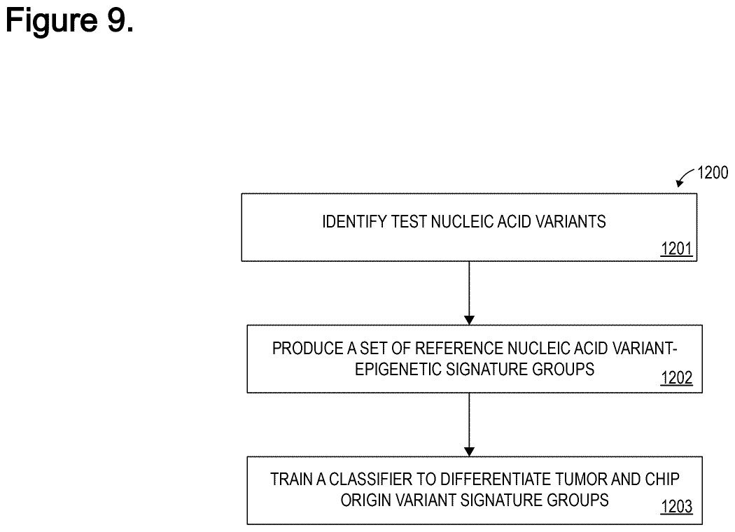

FIG. 6 is a flowchart illustrating an example training method 900 for generating the ML module 830 using the training module 820. The training module 820 can implement supervised, unsupervised, and/or semi-supervised (e.g., reinforcement based) machine learning-based classification models 840. The method 900 illustrated in FIG. 9 is an example of a supervised learning method; variations of this example of training method are discussed below, however, other training methods can be analogously implemented to train unsupervised and/or semi-supervised machine learning models.

The training method 900 may determine (e.g., access, receive, retrieve, etc.) data at step 910. The data may comprise tumor derived/non-tumor derived bodily fluid sample data. The data may comprise sequence data, epigenetic data, and/or fragmentomic data for one or more sequence fragments reads and/or variants, each sequence fragment/read and/or variant having an assigned tumor derived or non-tumor derived origin status.

The training method 900 may generate, at step 920, a training data set and a testing data set. The training data set and the testing data set may be generated by randomly assigning data to either the training data set or the testing data set. In some implementations, the assignment of computation parameters and associated experimental parameters as training or testing data may not be completely random. As an example, a majority of the computation parameters and associated experimental parameters may be used to generate the training data set. For example, 75% of the computation parameters and associated experimental parameters may be used to generate the training data set and 25% may be used to generate the testing data set. In another example, 80% of the computation parameters and associated experimental parameters may be used to generate the training data set and 20% may be used to generate the testing data set.

The training method 900 may determine (e.g., extract, select, etc.), at step 930, one or more features that can be used by, for example, a classifier to differentiate among different classification of tumor derived vs. non-tumor derived status. As an example, the training method 900 may determine a set of features from the tumor derived/non-tumor derived bodily fluid sample data. In a further example, a set of features may be determined from data that is different than the tumor derived/non-tumor derived bodily fluid sample data in either the training data set or the testing data set. Such other data may be used to determine an initial set of features, which may be further reduced using the training data set.

The training method 900 may train one or more machine learning models using the one or more features at step 940. In one example, the machine learning models may be trained using supervised learning. In another example, other machine learning techniques may be employed, including unsupervised learning and semi-supervised. The machine learning models trained at 940 may be selected based on different criteria depending on the problem to be solved and/or data available in the training data set. For example, machine learning classifiers can suffer from different degrees of bias. Accordingly, more than one machine learning model can be trained at 940, optimized, improved, and cross-validated at step 950.

The training method 900 may select one or more machine learning models to build a predictive model at 960. The predictive model may be evaluated using the testing data set. The predictive model may analyze the testing data set and generate predicted tumor/non-tumor origin statuses at step 970. Predicted tumor/non-tumor origin may be evaluated at step 980 to determine whether such values have achieved a desired accuracy level. Performance of the predictive model may be evaluated in a number of ways based on a number of true positives, false positives, true negatives, and/or false negatives classifications of the plurality of data points indicated by the predictive model.

For example, the false positives of the predictive model may refer to a number of times the predictive model incorrectly classified a sequence fragment/read and/or variant as tumor origin that was in reality non-tumor origin. Conversely, the false negatives of the predictive model may refer to a number of times the machine learning model classified a sequence fragment/read and/or variant as non-tumor origin when, in fact, the sequence fragment/read and/or variant was tumor origin. True negatives and true positives may refer to a number of times the predictive model correctly classified one or more sequence fragment/read and/or variant. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives. When such a desired accuracy level is reached, the training phase ends and the predictive model (e.g., the ML module 830) may be output at step 990; when the desired accuracy level is not reached, however, then a subsequent iteration of the training method 900 may be performed starting at step 910 with variations such as, for example, considering a larger collection of data.

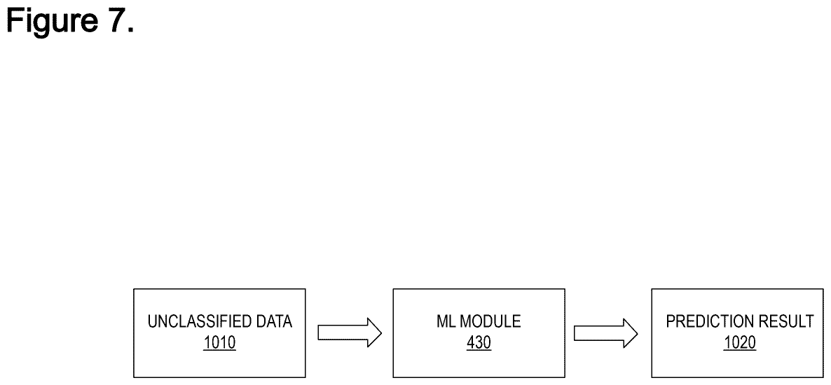

FIG. 6 is an illustration of an exemplary process flow for using a machine learning-based classifier to classify a sequence fragment/read and/or variant as tumor origin or non-tumor origin. As illustrated in FIG. 7, sequence data, epigenetic data, and/or fragmentomic data for an unclassified sequence fragment/read and/or variant 1010 may be provided as input to the ML module 830. The ML module 830 may process the sequence data, epigenetic data, and/or fragmentomic data for the unclassified sequence fragment/read and/or variant 1010 using a machine learning-based classifier(s) to arrive at a prediction result 1020. The prediction result 1020 may identify one or more characteristics of the sequence data, epigenetic data, and/or fragmentomic data for an unclassified sequence fragment/read and/or variant 1010. For example, the classification result 1020 may identify the origin status of the sequence fragment/read and/or variant 1010 (e.g., whether the sequence fragment/read and/or variant is tumor origin or non-tumor origin). Thus, in an embodiment, disclosed is a method implemented using a network-based computer system comprising one or more processors, a network interface, and one or more memories, the method comprising retrieving, by the computer system, sequence data, epigenetic data, and/or fragmentomic data having an indicated tumor derived origin or non-tumor derived origin status; and training, by the one or more processors, a machine-learning model by fitting one or more models to the sequence data, epigenetic data, and/or fragmentomic data, wherein each of the one or more models is configured to receive as input sequence data, epigenetic data, and/or fragmentomic data of an individual, and provide as output a prediction of the individual having or developing a tumor.

In some aspects, this disclosure provides methods of coupling somatic genomic information with epigenetic signatures (e.g. methylation profiles, fragmentomics, etc.) which provide additional genomic signal to aid in the bioinformatic exclusion of background clonal hematopoiesis of indeterminate potential (CHIP) variants to deterministically call tumor or in known CHIP genes. In some embodiments, the methylation and fragmentation profiles of normal white blood cells exhibiting CHIP are differentiated from their pathogenic tumor counterparts. In certain embodiments, incorporation of targeted hybridization panels investigating known methylation sites or other epigenetic sites in genes of likely CHIP interference (e.g., DNMT3A, TP53, LRP1B, KRAS, etc.) in the NGS workflow provides orthogonal information to adjudicate CHIP. Similarly, incorporation of bioinformatic modules analyzing the ctDNA fragment distribution of genes known to exhibit a high prevalence CHIP are used as orthogonal information to generate CHIP adjudication callers in some embodiments. The combination of known CHIP prevalence genes or other genomic regions and epigenetic profiles (e.g., methylation profiles, ctDNA fragment distributions (e.g., fragmentomics), bi-sulfide sequencing, and/or the like) provide technological solutions to improve the efficacy of diagnostics.

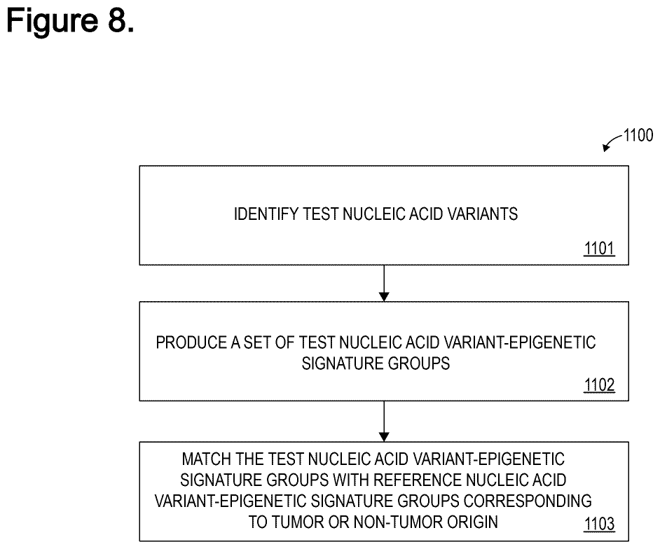

To illustrate, FIG. 8 is a flow chart that schematically depicts exemplary method steps of differentiating tumor and clonal hematopoiesis of indeterminate potential (CHIP) origin nucleic acid variants from one another in a test sample obtained from a test subject using a computer according to some embodiments of the invention. As shown, method 1100 includes identifying nucleic acid variants in a set of targeted genomic regions from sequence information obtained from nucleic acids in the test sample to produce a set of identified test nucleic acid variants (step 1101). The method also includes identifying at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups (step 1102). In some embodiments, the epigenetic signature, for example—methylation signature, may be determined based on the methods and systems disclosed in PCT Application No. PCT/US2021/025201. The method also includes matching given test nucleic acid variant-epigenetic signature groups in the set of test nucleic acid variant-epigenetic signature groups with reference nucleic acid variant-epigenetic signature groups corresponding to tumor origin nucleic acid variants or with reference nucleic acid variant-epigenetic signature groups corresponding to CHIP origin nucleic acid variants, thereby differentiating the tumor and the CHIP origin nucleic acid variants from one another in the test sample obtained from the test subject (step 1103). In some embodiments, method 1100 also includes using at least one trained classifier to differentiate tumor and CHIP origin nucleic acid variants from one another in the set of test nucleic acid variant-epigenetic signature groups to produce a set of differentiated tumor and CHIP origin nucleic acid variants present in the test sample. In some embodiments, the method also includes administering at least one therapy to the test subject based upon one or more of the differentiated tumor origin nucleic acid variants in the set of differentiated tumor and CHIP origin nucleic acid variants present in the test sample to thereby treat the cancer in the test subject.

To illustrate, FIG. 9 is a flow chart that schematically depicts exemplary method steps of generating a trained classifier using a computer according to some embodiments of the invention. As shown, method 1200 includes identifying nucleic acid variants in at least one set of targeted genomic regions from sequence information obtained from nucleic acids in a plurality of reference samples to produce a set of identified reference nucleic acid variants (step 1201). Method 1200 also includes identifying at least one epigenetic signature corresponding to a given nucleic acid variant for a plurality of the identified reference nucleic acid variants in the set of identified reference nucleic acid variants from epigenetic information obtained from the nucleic acids in the reference samples to produce a set of reference nucleic acid variant-epigenetic signature groups (step 1202). Method 1200 also includes training a machine learning algorithm using at least a portion of the set of reference nucleic acid variant-epigenetic signature groups to create at least one trained classifier that is configured to classify one or more test nucleic acid variant-epigenetic signature groups as comprising tumor and/or clonal hematopoiesis of indeterminate potential (CHIP) origin nucleic acid variants (step 1203).

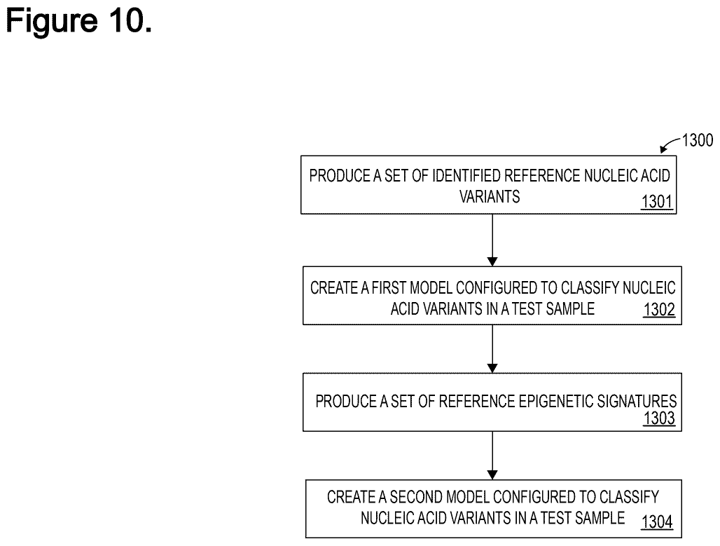

To further illustrate, FIG. 10 is a flow chart that schematically depicts exemplary method steps of generating a trained classifier using a computer according to some embodiments of the invention. As shown, method 1300 includes identifying nucleic acid variants in at least one set of targeted genomic regions from sequence information obtained from nucleic acids in a plurality of reference samples to produce a set of identified reference nucleic acid variants (step 1301). Method 1300 also includes training a machine learning algorithm using at least a portion of the set of identified reference nucleic acid variants to create at least a first model that is configured to classify nucleic acid variants in the set of targeted genomic regions from sequence information obtained from nucleic acids in a test sample to produce a set of identified test nucleic acid variants (step 1302). Method 1300 also includes identifying at least one epigenetic signature corresponding to a given nucleic acid variant for a plurality of the reference identified nucleic acid variants in the set of identified reference nucleic acid variants from epigenetic information obtained from the nucleic acids in the reference samples to produce a set of reference epigenetic signatures (step 1303). Method 1300 also includes training the machine learning algorithm using at least a portion of the set of reference epigenetic signatures to create at least a second model that is configured to differentiate tumor and clonal hematopoiesis of indeterminate potential (CHIP) origin nucleic acid variants from one another in the set of test nucleic acid variant-epigenetic signature groups to produce a set of identified test nucleic acid variants (step 1304).

In some embodiments, the set of test nucleic acid variant-epigenetic signature groups comprises at least first and second members that include identical nucleic acid variants and different corresponding epigenetic signatures. In some of these embodiments, the different corresponding epigenetic signatures include differing epigenetic states or statuses exhibited by one or more epigenetic loci in a given targeted genomic region. In some of these embodiments, the different corresponding epigenetic signatures include differing cell-free nucleic acid (cfNA) fragment length, position, and/or endpoint density distributions. In some embodiments, the set of test nucleic acid variant-epigenetic signature groups includes at least first and second members that include different nucleic acid variants and identical corresponding epigenetic signatures.

In some embodiments, the matching step includes using at least one trained classifier to differentiate the tumor and the CHIP origin nucleic acid variants from one another in the test sample obtained from the test subject. In some embodiments, the set of identified nucleic acid variants includes somatic nucleic acid variants. In some embodiments, a given targeted genomic region includes two or more nucleic acid variant loci. In some embodiments, the set of test nucleic acid variant-epigenetic signature groups includes at least one member that includes one or more nucleic acid variants and one or more corresponding epigenetic signatures that are from different genomic regions in the set of set of targeted genomic regions. In some embodiments, the set of test nucleic acid variant-epigenetic signature groups comprises at least one member that comprises one or more nucleic acid variants and one or more corresponding epigenetic signatures that are in an identical genomic region in the set of set of targeted genomic regions. In some embodiments, the plurality of targeted genomic regions comprise one or more genes selected from the group consisting of: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIFI, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET. In some embodiments, the nucleic acids in the sample comprise cell-free nucleic acid (cfNA) fragments and/or nucleic acid molecules obtained from one or more tissues or cells in the sample. In some embodiments, the epigenetic signature comprises a cfNA fragment length, position, and/or endpoint density distribution.

In some embodiments, the epigenetic signature comprises an epigenetic state or status exhibited by one or more epigenetic loci in a given targeted genomic region. In some embodiments, the epigenetic state or status comprises a presence or absence of methylation, hydroxymethylation, acetylation, ubiquitylation, phosphorylation, sumoylation, ribosylation, citrullination, and/or a histone post-translational modification or other histone variation. In some embodiments, the method further includes disregarding differentiated CHIP origin nucleic acid variants from further analysis. In some embodiments, the method further includes generating at least one report that lists the tumor and CHIP origin nucleic acid variants differentiated from one another in the test sample.

In some embodiments, the method further includes identifying at least one cancer type associated with the differentiated tumor origin nucleic acid variants. In some embodiments, the method further includes administering at least one therapy to the test subject to treat the identified cancer type. In some embodiments, the method further includes administering at least one therapy to the test subject based upon one or more of the differentiated tumor origin nucleic acid variants. In some embodiments, one or more cells comprise the nucleic acids in the test sample.

In some embodiments, the method further includes identifying, by the computer, nucleic acid variants in the set of targeted genomic regions from sequence information obtained from nucleic acids in a test sample obtained from a test subject to produce a set of identified test nucleic acid variants, identifying, by the computer, at least one epigenetic signature corresponding to a given test nucleic acid variant for a plurality of the identified test nucleic acid variants in the set of identified test nucleic acid variants from epigenetic information obtained from the nucleic acids in the test sample to produce a set of test nucleic acid variant-epigenetic signature groups, and using the trained classifier to differentiate the tumor and the CHIP origin nucleic acid variants in the set of test nucleic acid variant-epigenetic signature groups from one another in the test sample obtained from the test subject. In some embodiments, the second model is a further trained version of the first model. In some embodiments, the set of reference nucleic acid variant-epigenetic signature groups comprises prevalence data for epigenetic signatures corresponding to given nucleic acid variants in the set of identified reference nucleic acid variants.

In some embodiments, identifying the at least one epigenetic signature corresponding to a given nucleic acid variant comprises: determining epigenetic rates corresponding to the given nucleic acid variant, wherein at least a first epigenetic rate is generated from a first sample obtained from a given subject at a first time point, and at least a second epigenetic rate is generated from a second sample obtained from the given subject at a second time point that differs from the first time point; adjusting at least one epigenetic rate threshold based on at least the first epigenetic rate to produce an adjusted epigenetic rate threshold; and using the adjusted epigenetic rate threshold to identify the epigenetic signature. In some embodiments, the first and second sample samples comprise test samples. In some embodiments, the first and second sample samples comprise reference samples. In some embodiments, the first sample comprises a tumor tissue sample. In some embodiments, the second sample comprises a bodily fluid sample. Some embodiments include using epigenetic rates to identify tumor fractions in samples. Certain embodiments optionally include determining a plurality of epigenetic rates for a plurality of genomic regions of a first sample; determining a likelihood of a tumor fraction for one or more of the plurality of genomic regions in a second sample based on a predetermined set of epigenetic rates of the plurality of genomic regions of the second sample, a set of epigenetic characteristics for a set of cell-free polynucleotides in the second sample mapped to the plurality of genomic regions, and the epigenetic rates of the plurality of genomic regions of the first sample; combining the plurality of likelihoods for one of more the plurality of genomic regions to determine an overall posterior probability for the presence of the cancer in the subject; and comparing the overall posterior probability for the presence of the cancer in the subject with a predetermined threshold. Some of these embodiments also include classifying a subject (a) as positive for circulating tumor DNA (ctDNA), if the overall posterior probability for the presence of the cancer in the subject is greater than or equal to the predetermined threshold, or (b) as negative for ctDNA, if the overall posterior probability for the presence of the cancer in the subject is less than the predetermined threshold. In some embodiments, the methods and systems used for analyzing the epigenetic status may be found in International Patent Application No. PCT/US2020/035605, entitled “METHODS AND SYSTEMS FOR IMPROVING PATIENT MONITORING AFTER SURGERY,” filed Jun. 1, 2020, which is incorporated by reference.

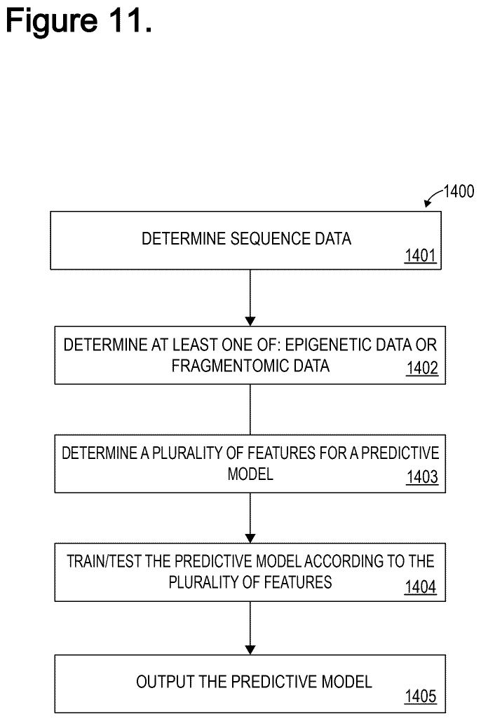

In an embodiment, shown in FIG. 11, a method 1400 for generating a predictive model is disclosed. In The method 1400 may be performed in whole or in part by a single computing device, a plurality of computing devices, and the like. The method 1400 may comprise determining sequence data at 1401. The method 1400 may comprise determining at least one of: epigenetic data or fragmentomic data at 1402. The method 1400 may comprise determining a plurality of features for a predictive model at 1403. The method 1400 may comprise training and/or testing the predictive model according to the plurality of features at 1404. The method 1400 may comprise outputting the predictive model at 1405.

The plurality of genomic regions may comprise at least one of: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIFI, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET. Determining sequence data may comprise obtaining a plurality of samples from a plurality of subjects, wherein the plurality of samples comprise a plurality of cell-free nucleic acids. The plurality of genomic regions may comprise at least one of: a genomic region known to be associated with a cancer type, a genomic region associated with a known methylation status, a genomic region known to be associated with hypomethylation, or a genomic region known to be associated with therapy response.

The epigenetic data may comprise at least one of: information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. Determining the epigenetic data associated with the plurality of sequence fragments comprises determining a methylation state of the plurality of sequence fragments.

Determining the methylation state of the plurality of sequence fragments may comprise determining at least one of: a methylation state vector or a methylated CpG density. Determining the methylation state vector may comprise aligning the plurality of sequence reads to a reference sequence, determining, based on the aligning, a methylation status of one or more CpG sites in a sequence read of the plurality of sequence reads and a location of the one or more CpG sites, and vectorizing the methylation status of the one or more CpG sites and the locations of the one or more CpG sites to generate the methylation state vector for the sequence read of the plurality of sequence reads. Determining the methylated CpG density may comprise aligning the plurality of sequence reads to a reference sequence, determining, based on the aligning, a methylation status of one or more CpG sites in a sequence read of the plurality of sequence reads, determining, based on the methylation status of the one or more CpG sites in the sequence read, that the sequence read is methylated or unmethylated, determining, for the plurality of sequence reads, a count of methylated sequence reads and a count of unmethylated sequence reads, and determining, based on the count of methylated sequence reads and the count of unmethylated sequence reads, the methylated CpG density.

The fragmentomic data may comprise at least one of: information regarding fragment size, nucleotide motifs at fragment ends, single-stranded jagged ends, genomic location of center point of the fragment length, genomic locations of fragment endpoints, genomic locations of fragment endpoints and/or any value indicating the endpoints of the fragment. Determining the fragmentomic data associated with the plurality of sequence fragments may comprise at least one of: determining a size of a sequence fragment of the plurality of fragments or determining an amount of the plurality of sequence fragments that have a particular size. The particular size may be a range. The range may be at least one of: 50-80, 50-100, 50-150, 100-150, 100-200, 150-200, 150-230, 200-300, or 300-400 bases.

Determining the fragmentomic data associated with the plurality of sequence fragments may comprise determining an end motif for the plurality of sequence fragments, wherein the end motif relates to an ending sequence of a sequence fragment. Determining the end motif for the plurality of sequence fragments may comprise aligning the plurality of sequence reads sequenced from the plurality of sequence fragments to a reference sequence and determining, based on the aligning, an end motif for each end of a sequence fragment of the plurality of sequence fragments. The ending sequences may comprise a number of bases, wherein the number of bases is between 1-6 bases. The ending sequences may comprise a number of bases that extends past the sequence fragment, wherein the number of bases is between 1-6 bases. The method 1400 may further comprise determining a frequency of occurrence of the end motif within the plurality of sequence fragments. The method 1400 may further comprise determining an end base of the end motif and determining a frequency of occurrence of the end base of the end motif. Determining the fragmentomic data associated with the plurality of sequence fragments may comprise determining a jagged end of a sequence fragment of the plurality of sequence fragments. Determining the jagged end of the sequence fragment of the plurality of sequence fragments may comprise determining an overhang index. The sequence fragment may be double-stranded with a first strand having a first portion and a second strand and determining the overhang index may comprise determining a methylation status of the first strand or the second strand that is proportional to a length of the first strand that overhangs the second strand and determining, based on the methylation status, the overhang index, wherein the overhang index provides a measure that a strand overhangs another strand.

Determining the fragmentomic data associated with the plurality of sequence fragments may comprise determining a genomic location of fragment endpoints. Determining the genomic location of fragment endpoints may comprise determining a windowed protection score (WPS). Determining the WPS may comprise determining a number of sequence fragments spanning a window and adjusting, based on any sequence fragments that start within the window, the number of sequence fragments spanning the window.

The method 1400 may further comprise determining an origin of a sequence fragment and assigning the origin of the sequence fragment to the sequence data, the epigenetic data, and the fragmentomic data associated with the sequence fragment. The origin may be tumor-derived or non-tumor derived, the origin is a tissue type, or the origin is a cancer type.

Determining, based on the at least the portion of the sequence data and the at least the portion of the at least one of: the epigenetic data or fragmentomic data, the plurality of features for the predictive model may comprise determining at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores and determining which, alone or in combination, of the at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores, have predictive value associated with an origin of a sequence fragment.

Training, based on the first portion of the sequence data and the at least one of: the epigenetic data or fragmentomic data, the predictive model according to the plurality of features may comprise training the predictive model according to a machine learning approach. The machine learning approach may comprise at least one of: a discriminant analysis, a decision tree, a nearest neighbor (NN) algorithm, a Bayesian network, a clustering algorithm, a neural network, a support vector machine (SVM), a logistic regression algorithm, a linear regression algorithms, a Markov model, or a principal component analysis (PCA). Testing, based on the second portion of the sequence data and the at least one of: the epigenetic data or fragmentomic data, the predictive model, may comprise causing the predictive model to be retrained.

The method 1400 may further comprise determining, for a subject, test sequence data comprising a plurality of sequence fragments associated with the plurality of genomic regions, wherein the plurality of sequence fragments are sequenced from a sample from the subject, determining at least one of: test epigenetic data or test fragmentomic data associated with the plurality of sequence fragments, providing, to the predictive model, test sequence data, test epigenetic data, and test fragmentomic data of the subject, and determining, based on the test sequence data, the test epigenetic data, and the test fragmentomic data of the subject, an origin of at least on sequence fragment in the sequence data. The origin may be one of tumor derived or non-tumor derived.

The method 1400 may further comprise administering one or more therapies to the subject based on the origin being tumor derived. The therapies may comprise administering chemotherapy, administering radiation therapy, or performing surgery to resect all or a portion of a tumor. The therapies may comprise administering at least one of: ALECENSA®, ALUNBRIG®, BRAFTOVI®, ERBITUX®, GAVRETO™, GILOTRIF®, HERCEPTIN®, IRESSA®, KADCYLA®, KEYTRUDA®, LORBRENA®, LUMAKRAS™, LYNPARZA®, MEKINIST®, OPDIVO®, PERJETA®, PIQRAY®, RETEVMO™, ROZLYTREK™, RUBRACA®, TABRECTA™, TAFINLAR®, TAGRISSO®, TALZENNA®, TARCEVA®, TEPMETKO™, TYKERB®, VITRAKVI®, VIZIMPRO®, XALKORI®, YBREVANT™, YERVOY®, or ZYKADIA®.

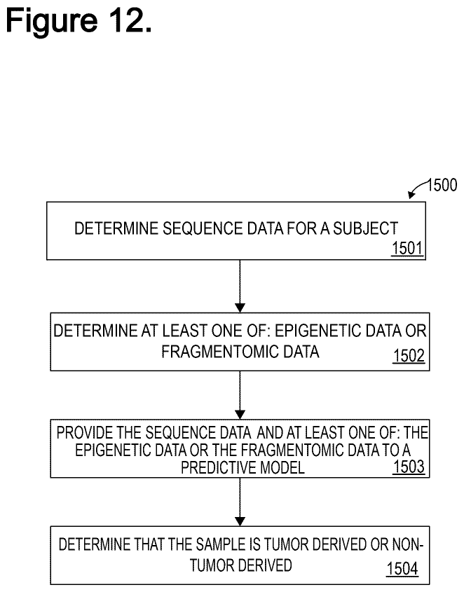

In an embodiment, shown in FIG. 12, a method 1500 for determining an origin of a sample is disclosed. The method 1500 may be performed in whole or in part by a single computing device, a plurality of computing devices, and the like. The method 1500 may comprise determining sequence data for a sample of a subject at 1501. The method 1500 may comprise determining at least one of: epigenetic data or fragmentomic data at 1502. The method 1500 may comprise providing the sequence data and at least one of the epigenetic data or the fragmentomic data to a predictive model. The method 1500 may comprise determining, based on the predictive model, that the sample is tumor derived or non-tumor derived. The method 1500 may further comprising generating the predictive model. Generating the predictive model may comprise determining sequence data of a plurality of sequence fragments associated with a plurality of genomic regions, wherein the sequence data comprises a plurality of sequence reads, wherein the plurality of sequence reads are sequenced from the plurality sequence fragments from a plurality of samples, wherein each sample of the plurality of samples is labeled as a tumor derived or a non-tumor derived, determining at least one of: epigenetic data or fragmentomic data associated with the plurality of sequence fragments, determining, based on at least a portion of the sequence data and at least a portion of at least one of: the epigenetic data or fragmentomic data, a plurality of features for the predictive model, training, based on a first portion of the sequence data and at least one of: the epigenetic data or fragmentomic data, the predictive model according to the plurality of features, testing, based on a second portion of the sequence data and at least one of: the epigenetic data or fragmentomic data, the predictive model, and outputting, based on the testing, the predictive model.

The plurality of genomic regions may comprise at least one of: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIFI, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET.

Determining sequence data may comprise obtaining a plurality of samples from a plurality of subjects, wherein the plurality of samples comprise a plurality of cell-free nucleic acids. The plurality of genomic regions may comprise at least one of: a genomic region known to be associated with a cancer type, a genomic region associated with a known methylation status, a genomic region known to be associated with hypomethylation, or a genomic region known to be associated with therapy response.

The epigenetic data may comprise at least one of: information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. Determining the epigenetic data associated with the plurality of sequence fragments may comprise determining a methylation state of the plurality of sequence fragments. Determining the methylation state of the plurality of sequence fragments may comprise determining at least one of: a methylation state vector or a methylated CpG density. Determining the methylation state vector may comprise aligning the plurality of sequence reads to a reference sequence, determining, based on the aligning, a methylation status of one or more CpG sites in a sequence read of the plurality of sequence reads and a location of the one or more CpG sites, and vectorizing the methylation status of the one or more CpG sites and the locations of the one or more CpG sites to generate the methylation state vector for the sequence read of the plurality of sequence reads. Determining the methylated CpG density may comprise aligning the plurality of sequence reads to a reference sequence, determining, based on the aligning, a methylation status of one or more CpG sites in a sequence read of the plurality of sequence reads, determining, based on the methylation status of the one or more CpG sites in the sequence read, that the sequence read is methylated or unmethylated, determining, for the plurality of sequence reads, a count of methylated sequence reads and a count of unmethylated sequence reads, and determining, based on the count of methylated sequence reads and the count of unmethylated sequence reads, the methylated CpG density.

The fragmentomic data may comprise at least one of: information regarding fragment size, nucleotide motifs at fragment ends, single-stranded jagged ends, genomic location of center point of the fragment length, genomic locations of fragment endpoints, genomic locations of fragment endpoints and/or any value indicating the endpoints of the fragment. Determining the fragmentomic data associated with the plurality of sequence fragments may comprise at least one of: determining a size of a sequence fragment of the plurality of fragments or determining an amount of the plurality of sequence fragments that have a particular size. The particular size may be a range. The range may be at least one of: 50-80, 50-100, 50-150, 100-150, 100-200, 150-200, 150-230, 200-300, or 300-400 bases. Determining the fragmentomic data associated with the plurality of sequence fragments may comprise determining an end motif for the plurality of sequence fragments, wherein the end motif relates to an ending sequence of a sequence fragment. Determining the end motif for the plurality of sequence fragments may comprise aligning the plurality of sequence reads sequenced from the plurality of sequence fragments to a reference sequence and determining, based on the aligning, an end motif for each end of a sequence fragment of the plurality of sequence fragments. The ending sequence may comprise a number of bases. The number of bases may be between 1-6 bases. The ending sequence comprises a number of bases that extends past the sequence fragment, wherein the number of bases is between 1-6 bases. The method 1500 may further comprise determining a frequency of occurrence of the end motif within the plurality of sequence fragments. The method 1500 may further comprise determining an end base of the end motif and determining a frequency of occurrence of the end base of the end motif.

Determining the fragmentomic data associated with the plurality of sequence fragments comprises determining a jagged end of a sequence fragment of the plurality of sequence fragments. Determining the jagged end of the sequence fragment of the plurality of sequence fragments comprises determining an overhang index. The sequence fragment may be double-stranded with a first strand having a first portion and a second strand and determining the overhang index may comprise determining a methylation status of the first strand or the second strand that is proportional to a length of the first strand that overhangs the second strand and determining, based on the methylation status, the overhang index, wherein the overhang index provides a measure that a strand overhangs another strand.

Determining the fragmentomic data associated with the plurality of sequence fragments may comprise determining a genomic location of fragment endpoints. Determining the genomic location of fragment endpoints may comprise determining a windowed protection score (WPS). Determining the WPS may comprise determining a number of sequence fragments spanning a window and adjusting, based on any sequence fragments that start within the window, the number of sequence fragments spanning the window.

The method 1500 may further comprise determining an origin of a sequence fragment and assigning the origin of the sequence fragment to the sequence data, the epigenetic data, and the fragmentomic data associated with the sequence fragment. The origin may be tumor-derived or non-tumor derived, the origin is a tissue type, or the origin is a cancer type.

Determining, based on the at least the portion of the sequence data and the at least the portion of the at least one of: the epigenetic data or fragmentomic data, the plurality of features for the predictive model comprises determining at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores and determining which, alone or in combination, of the at least one of: methylation state vectors, methylation densities, fragment sizes, fragment size distributions, end motifs, end motif frequencies, jagged end presence, overhang indexes, genomic location of center point of the fragment length, genomic locations of fragment endpoints, genomic locations of fragment endpoints, any value indicating the endpoints of the fragment, or windowed protection scores, have predictive value associated with an origin of a sequence fragment.

Training, based on the first portion of the sequence data and the at least one of: the epigenetic data or fragmentomic data, the predictive model according to the plurality of features may comprise training the predictive model according to a machine learning approach. The machine learning approach may comprise at least one of: a discriminant analysis, a decision tree, a nearest neighbor (NN) algorithm, a Bayesian network, a clustering algorithm, a neural network, a support vector machine (SVM), a logistic regression algorithm, a linear regression algorithms, a Markov model, or a principal component analysis (PCA). Testing, based on the second portion of the sequence data and the at least one of: the epigenetic data or fragmentomic data, the predictive model, may comprise causing the predictive model to be retrained.

The method 1500 may further comprise, based on the sample being tumor derived, administering one or more therapies to the subject based on the origin being tumor derived. The therapies may comprise administering chemotherapy, administering radiation therapy, or performing surgery to resect all or a portion of a tumor. The therapies may comprise administering at least one of: ALECENSA®, ALUNBRIG®, BRAFTOVI®, ERBITUX®, GAVRETO™, GILOTRIF®, HERCEPTIN®, IRESSA®, KADCYLA®, KEYTRUDA®, LORBRENA®, LUMAKRAS™, LYNPARZA®, MEKINIST®, OPDIVO®, PERJETA®, PIQRAY®, RETEVMO™, ROZLYTREK™, RUBRACA®, TABRECTA™, TAFINLAR®, TAGRISSO®, TALZENNA®, TARCEVA®, TEPMETKO™, TYKERB®, VITRAKVI®, VIZIMPRO®, XALKORI®, YBREVANT™, YERVOY®, or ZYKADIA®.

Further information can be found in PCT App. No. PCT/US2021/015837, PCT/US2021/047619, and PCT/US2021/021994, Fairchild et al., Science Trans. Med. (2023), each of which are fully incorporated by reference herein.

### EXAMPLES

### Example 1: Annotate Tumor Vs Non-Tumor SNV/Indels to Allow for Filtration of Non-Tumor Variants

As described, the presence of clonal hematopoiesis (CH) variants, and biological noise, due to aging and therapy has potential to confound biomarker interpretation.

Currently, comprehensive methods to filter out non-tumor variants require genotyping the white blood cell (WBC) fraction of the paired plasma sample, which is a costly, complicated workflow. Of interested is a plasma-only, bioinformatics solution to identify non-tumor variants is needed for accurate biomarker assessments in the cell-free DNA (cfDNA).

Sensitivity with respect to annotation of using buffy coat sequencing can impact the interpretation of clinical significance of variants, particularly in genes with mixed tumor/CH origin such as TP53 and ATM, as well as specificity of products that require CH filtration such as genomic MR and TMB score.

### Example 2: Study Design

To address the obstacles the Inventors identified variant calls obtained from >250,000 plasma samples comprising healthy donor, early and late-stage cancer patients sequenced on genomic and/or epigenomic liquid biopsy panels, further incorporating s public tissue datasets. The model was trained on paired plasma and WBC datasets and optimized with 10-fold cross-validation to produce a non-tumor and tumor variant classifier. Validation was provided with a cohort of paired plasma and WBC advanced cancer samples were genotyped on an epigenomic detection assay. An additional cohort of healthy donor samples, genotyped on the genomic detection assay was also assessed.

### Example 3: CHIP Genes

In some instances, somatic variants in these genes associated with hematological malignancies will be annotated as CHIP. These include ASXL1, DNMT3A, GNAS, JAK2, PPM1D, SF3B1, TET2, BCOR, CBL, CEBPA, CREBBP, ETV6, EZH2, FLT3, IDH1, IDH2, MPL, MYD88, NPM1, RUNX1, SH2B3, STAG2, STAT3, U2AF1, ZRSR2, PLCG2, CALR, CSF3R, DDX41, BRCC3, EZH1, KIR2DL1, KIR2DL3, KMT2C.

### Example 4: CHIP Variant List

In other instances, a variety of techniques may be implemented. One example includes logistic regression (LR) classifier using known biological features of CHIP trained on curated empirically determined datasets and external public datasets and applied to the larger empirically determined dataset for CHIP determination. Another example includes incorporating variants that have been identified in heme malignancies in COSMIC signatures in >2 samples, or similarly identified in heme malignancies in TCGA in >2 samples. Another example includes, LR classifier using proportion of heme samples as single feature for CHIP determination.

### Example 5: Additional Features

In some instances one can adjust annotation labels based on buffy coat by filtering undetected variants due to insufficient coverage for the sake of reducing tumor variants labeled CH. Another example would include expanding training dataset (additional internal/pharma samples, publicly available data), incorporate, assessment of tumor mutational burden (TMB) and genomic molecular response (MR) impact. Finally, utilization of methylation data into model training, including sample and/or variant level information, can be incorporated. As shown, these techniques involving aggregation, normalization, and encoding for feature determination, when applied for analysis leads to engineering of features which represent those of greatest importance. Ultimately such factors can be weighted in establishing a scoring metric for “CH burden”.

### Example 6: Findings

In accordance with embodiments described herein, an exemplary bioinformatic model is demonstrated that exhibits high sensitivity and specificity with WBC for discriminating tumor and non-tumor using only cfDNA. As shown herein, a bioinformatic model has improved sensitivity for identifying non-tumor variants over WBC sequencing at low VAFs (<0.6%).

In a paired plasma and WBC late stage cancer cohort, the majority of non-tumor variants were in known clonal hematopoiesis genes and variants of uncertain significance. No clinically actionable variants, except in ATM and CHEK2, were confirmed or annotated as non-tumor.

### Example 7: Multi-Feature Model

In accordance with embodiments described herein, one can generate a multi-feature model drawing upon data features of sources including variant-level information such as VAF, fragmentomics, methylation, and/or clinical test sample database, such as aggregated variant summary statistics from clinical patients, clonality, longitudinal variant variability, cancer-type variability, etc. and/or public data sets, such as COSMIC (variant frequency in cancer tissue), GnomAD (population-level allele frequencies), COSMIC mutational signatures (SBS signatures).

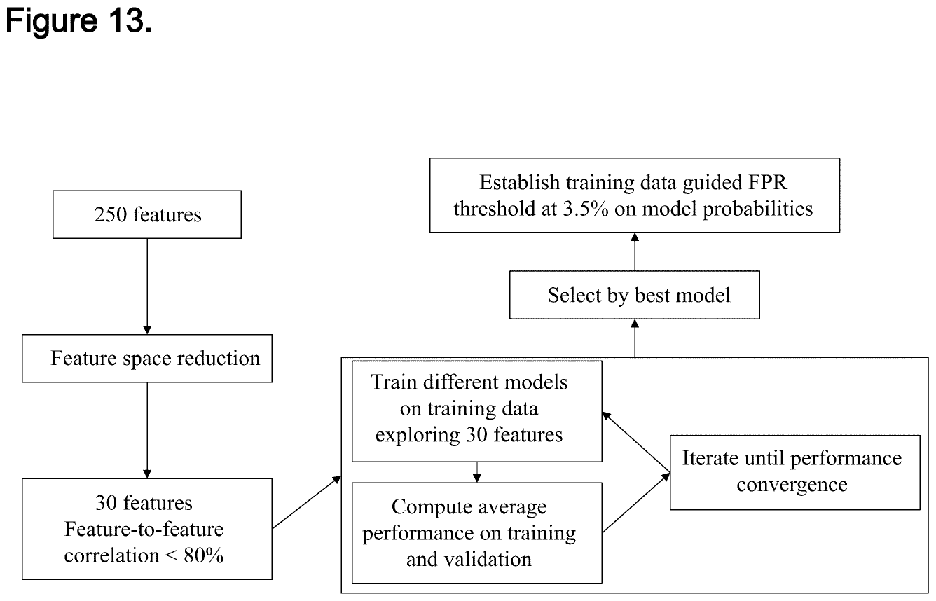

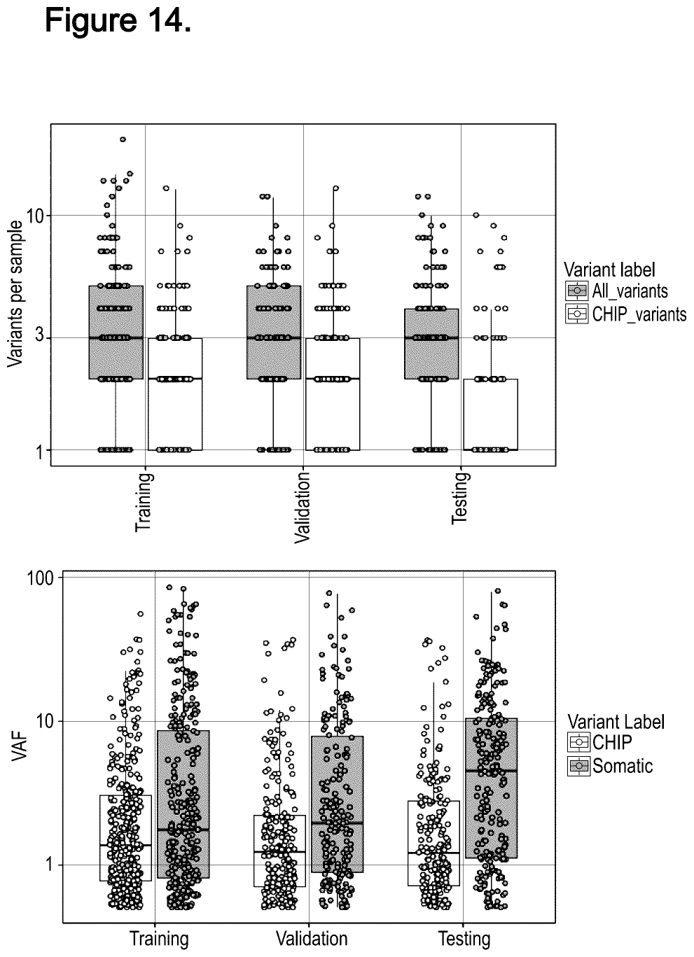

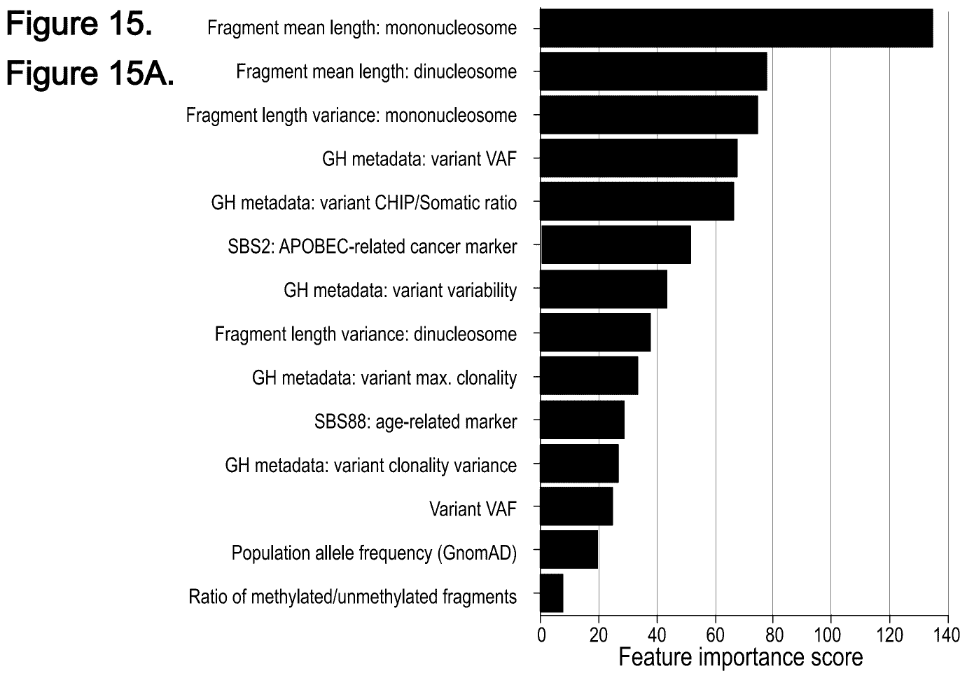

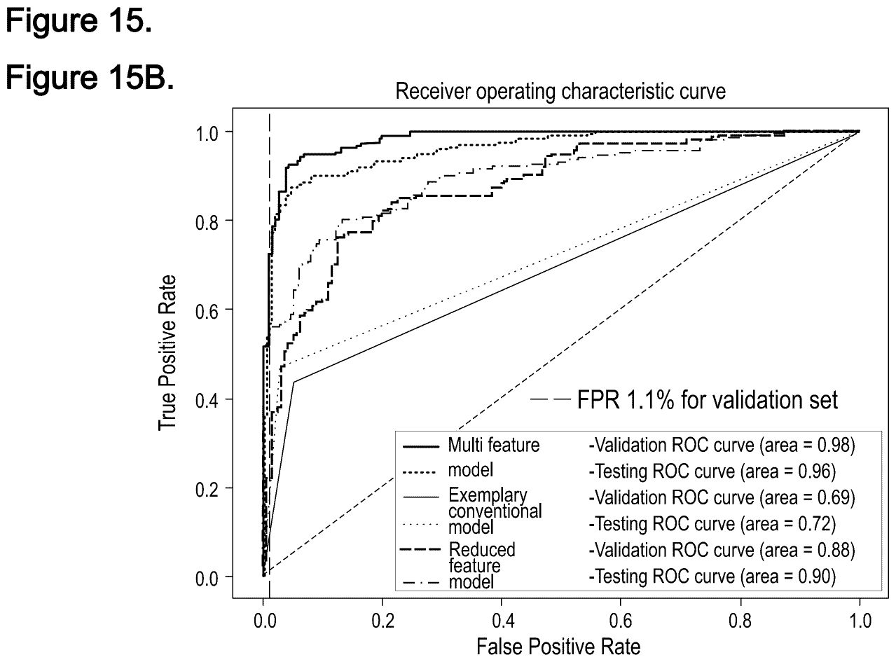

As shown in FIG. 13, A feature selection and model building process is depicted, with application of a correlation check that prevents correlation from exceeding a threshold (e.g., 70%, 80%, etc.) An example of ground truth state determined by matched buffy is shown in FIG. 14 for clinically relevant genes from NHC. Example of ground truth state determined by matched buffy. An example of a multi-feature model is shown in FIG. 15. Here, CHIP classifier exemplary multi-feature model uses 14 features and shows best performance in both validation and testing data. Exemplary sample level features depicted.

### Example 8: Multi-Feature Model Performance Characteristics, Buffy-Plasma Matched Samples

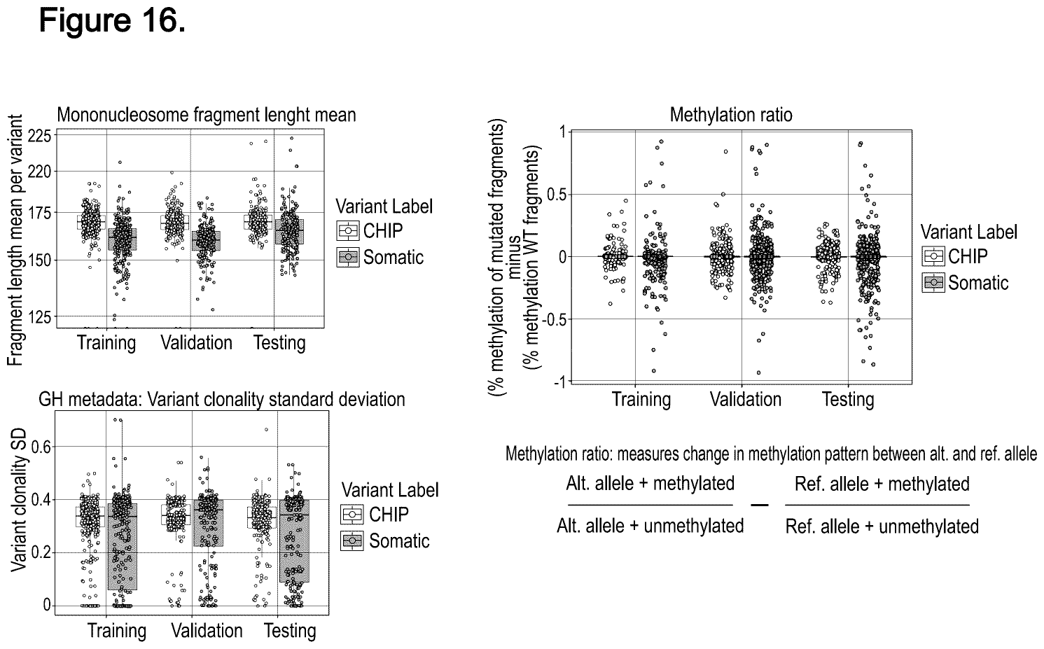

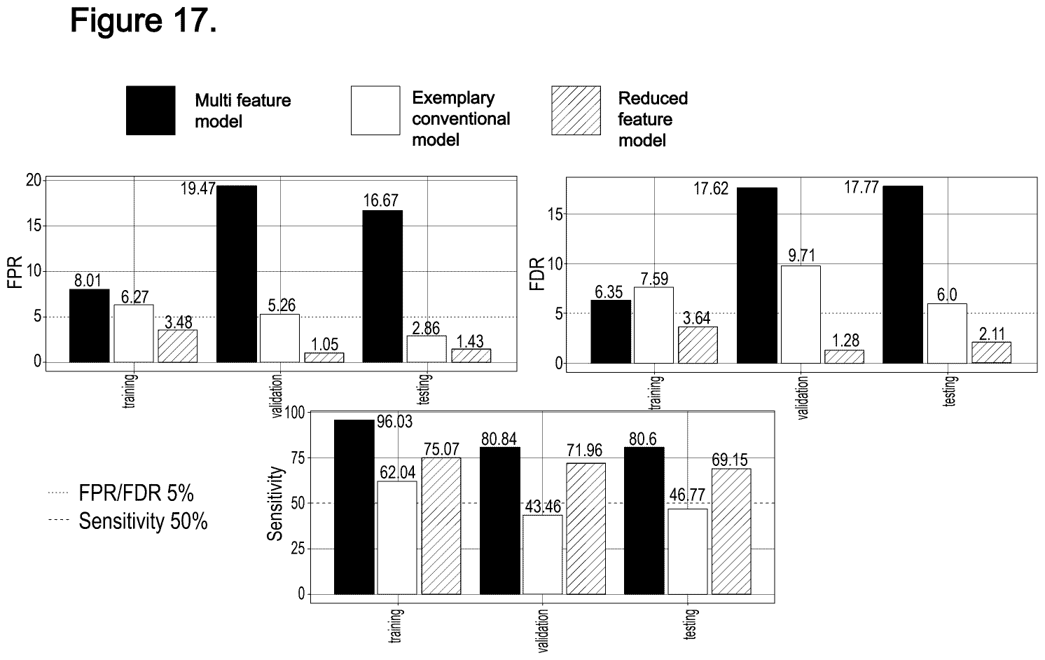

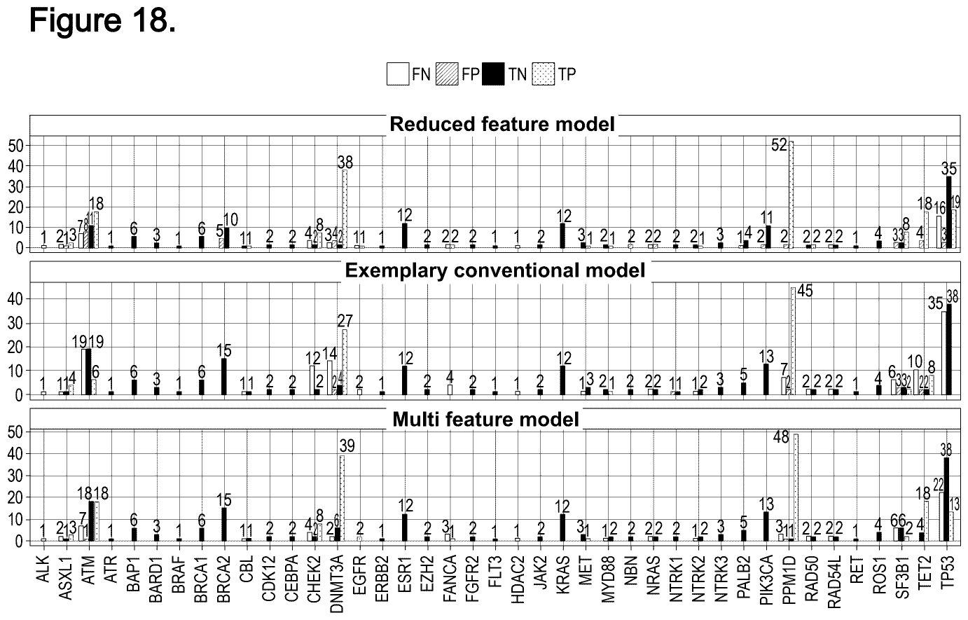

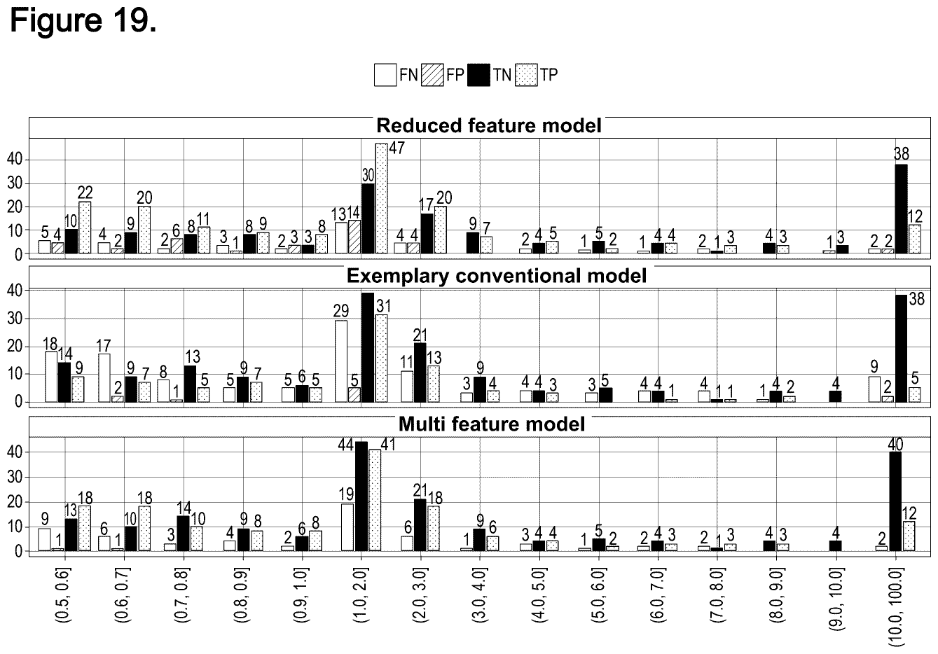

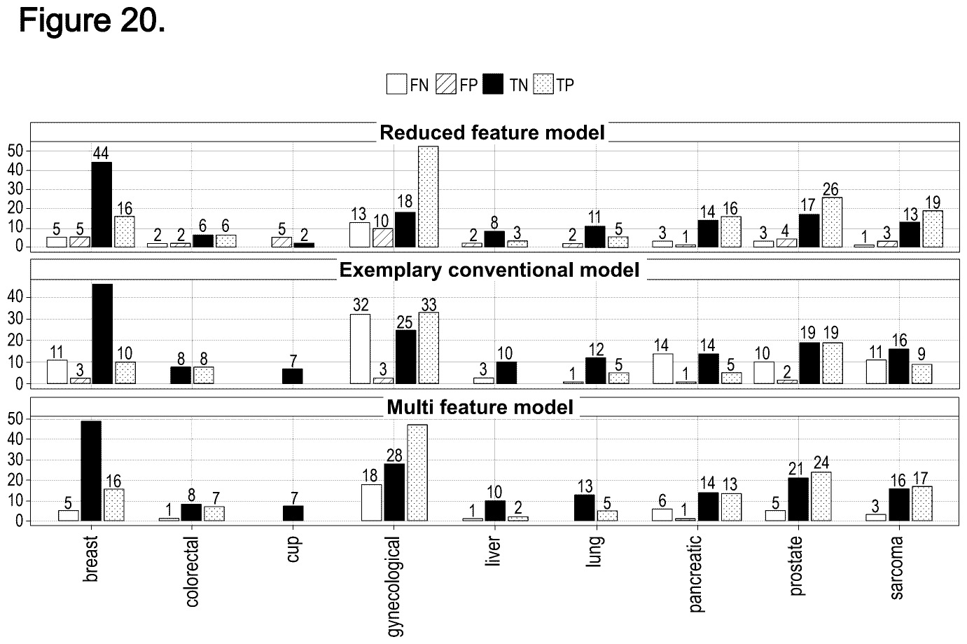

The described exemplary has superior receiver operator characteristics (ROC) as shown in FIG. 15B when compared to exemplary conventional model without multi-feature generation and reduced feature model obtained by CH variant labeling based on the presence of the same SNV/indel in paired cfDNA and buffy coat. As shown in FIG. 16, the CHIP classifier features can effectively capture nearly all differences between CHIP and somatic variants. As shown in FIG. 17, performance on clinically relevant genes in buffy-plasma matched samples, demonstrates superior performance for multi-feature model, exemplary conventional model without multi-feature generation and reduced feature model and as further shown in FIG. 18, no high FP genes detected in current classifier and as shown in FIG. 19. validation data using various VAF bins, demonstrate no high FP bins detected in exemplified multi-feature classifier and as shown in FIG. 20, for all recorded cancer types. No high FP cancer types were detected.

### Example 9: Multi-Feature Model Performance Characteristics, Tissue-Plasma Matched Samples

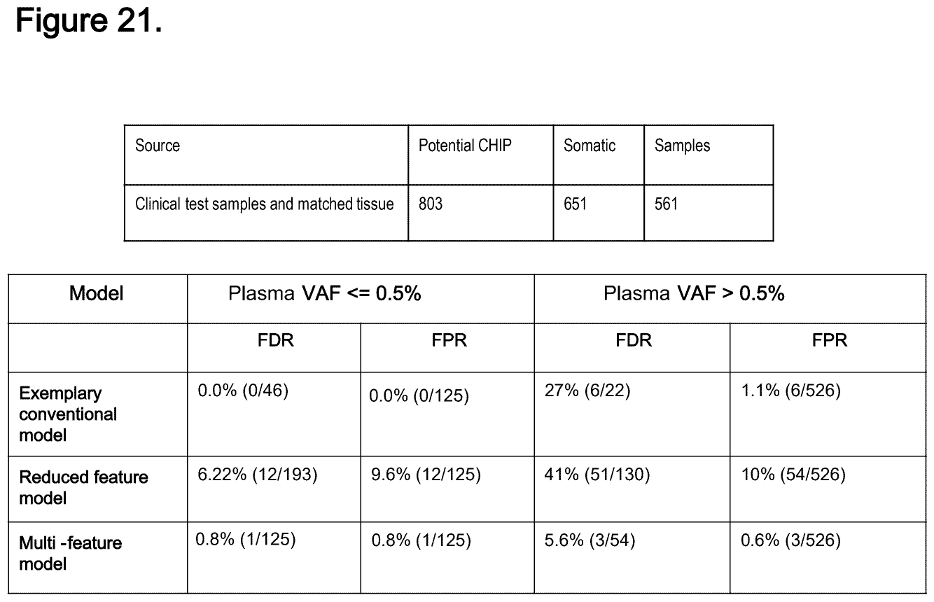

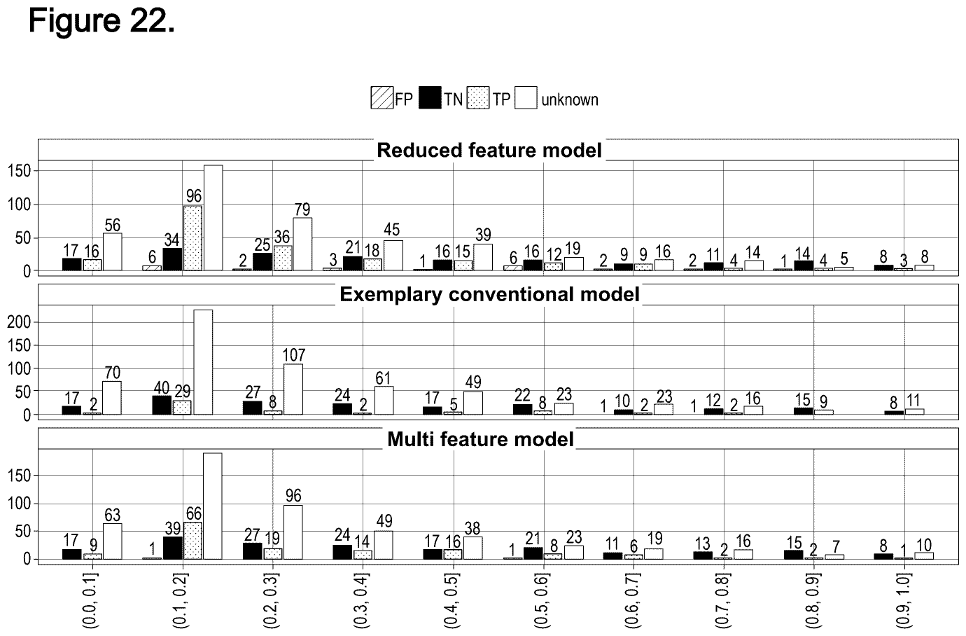

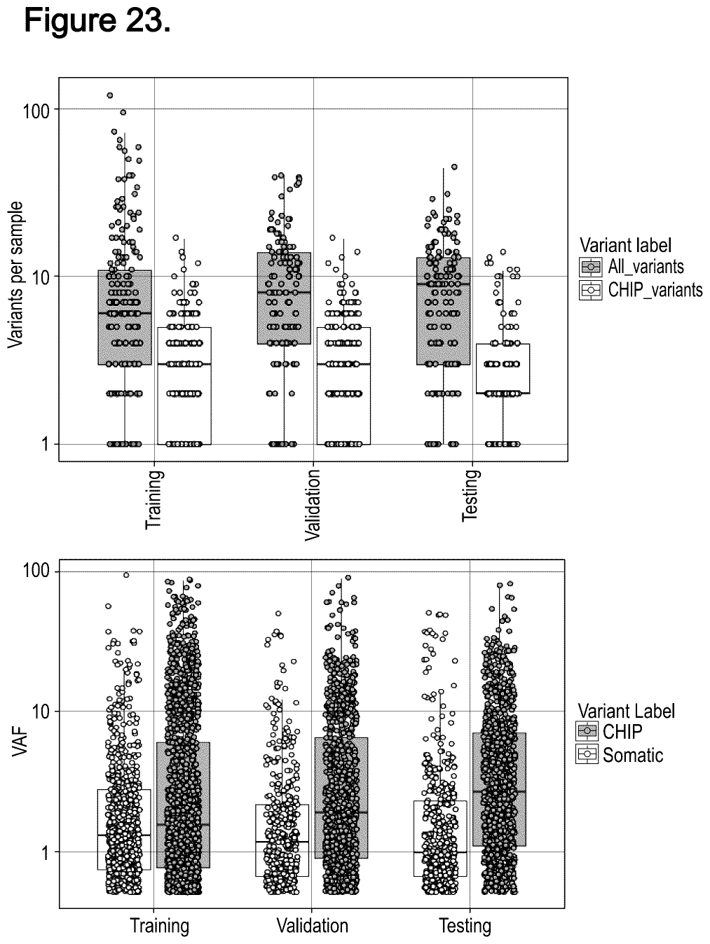

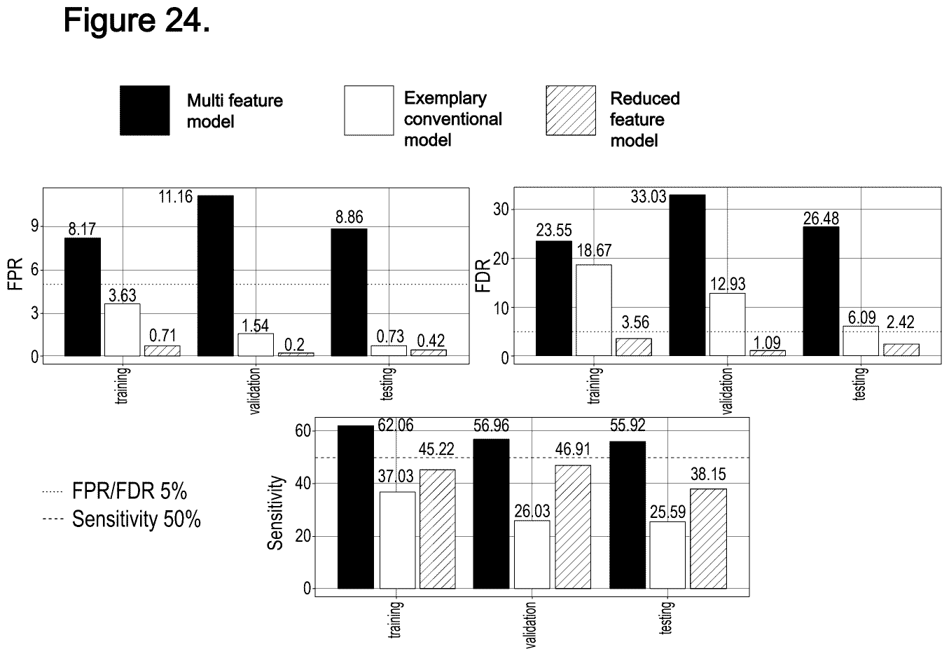

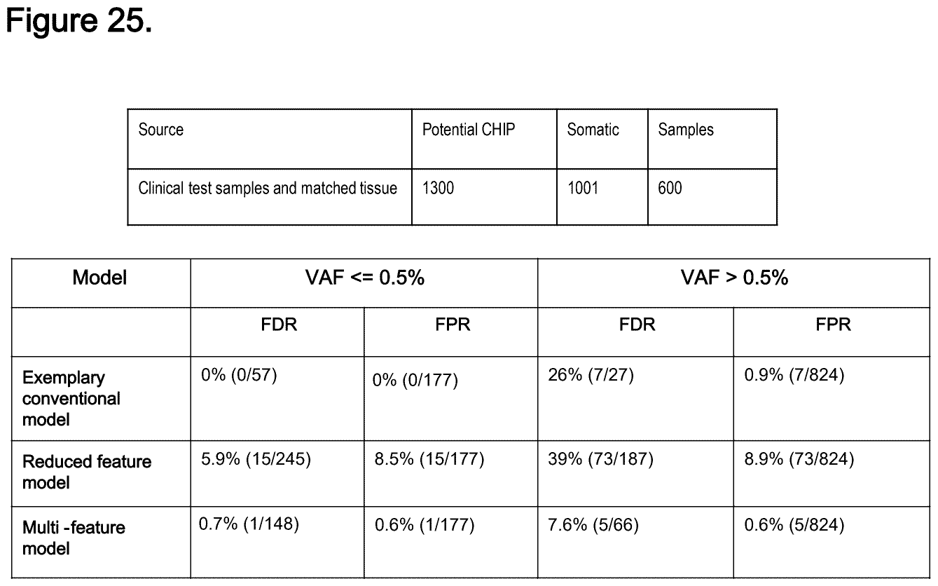

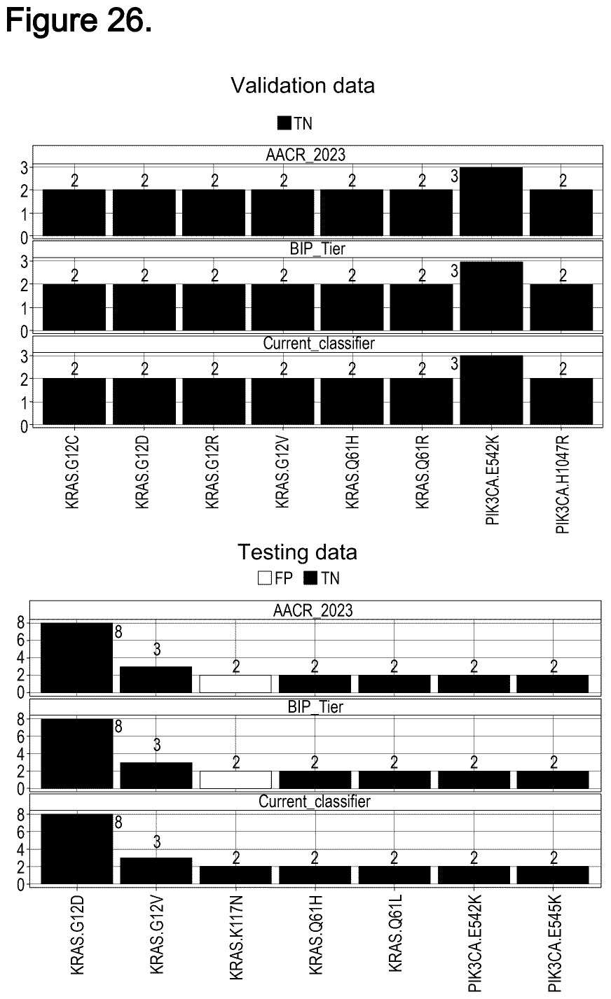

The described multi-feature model was then applied to tissue-plasma matched sample, whose superior performance is depicted in FIG. 21, and in FIG. 22. performance on clinically relevant genes: tissue-plasma matched samples, including specificity evaluation in low-VAF bins demonstrated the lack of high FP bins below 0.5% detected in current classifier. In FIG. 23, the exemplified multi-feature model was applied to a clinically relevant genes from exemplary panel of over 700 genes, a depiction of an example of ground truth state determined by matched buffy. It was observed that the multi-feature model possess superior performance on panel-wide variant as shown in FIGS. 24 and 25, when compared to exemplary conventional model without multi-feature generation and reduced feature model.

### Example 10: Multi-Feature Model Performance Characteristics, Actionable Variants

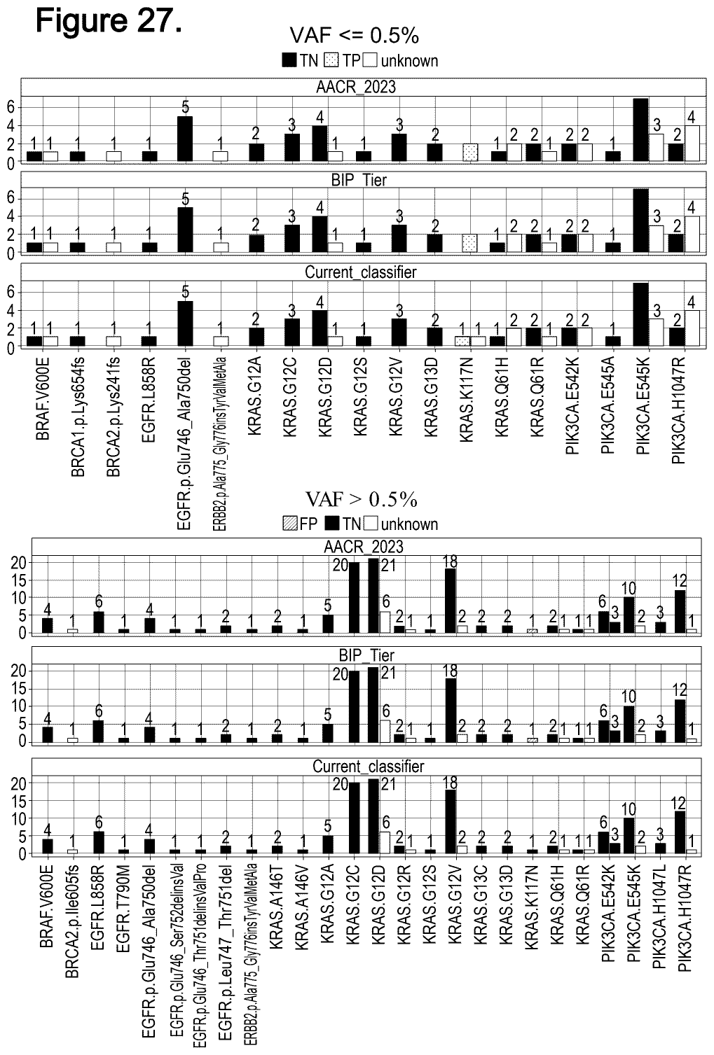

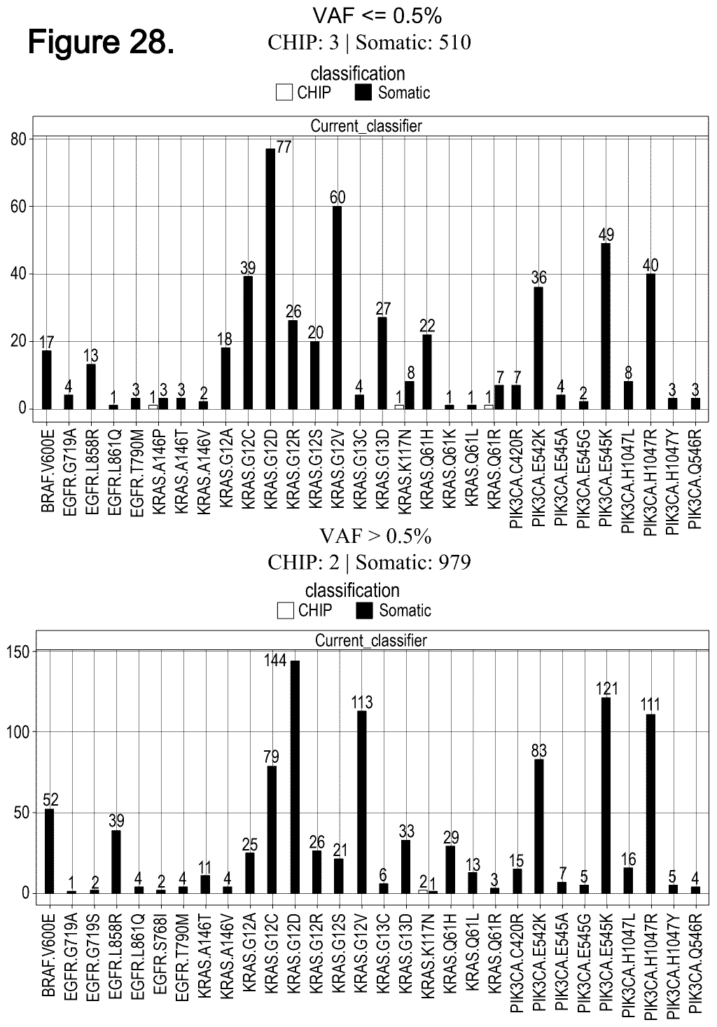

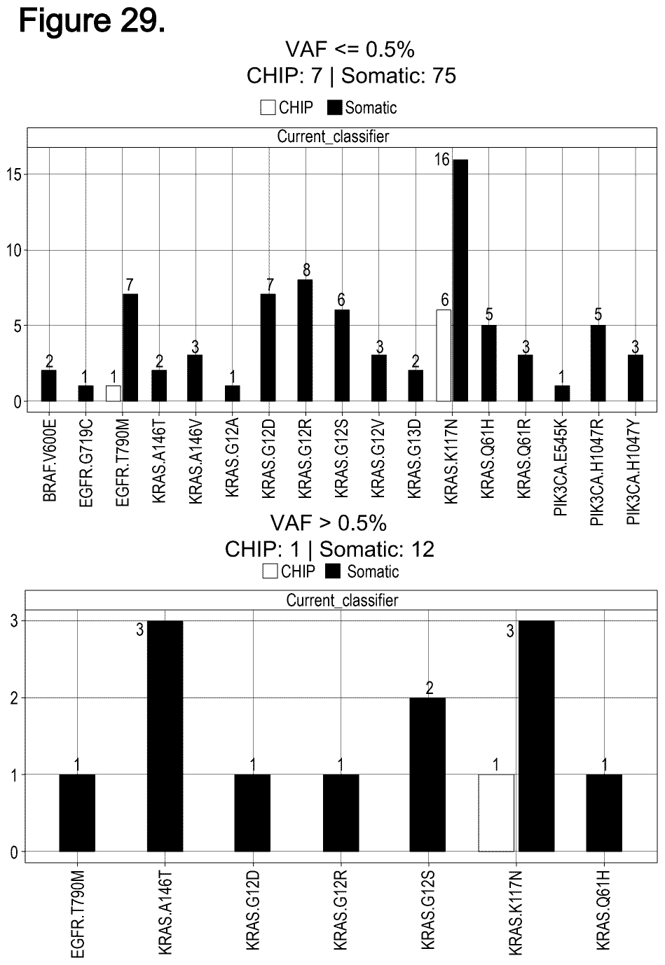

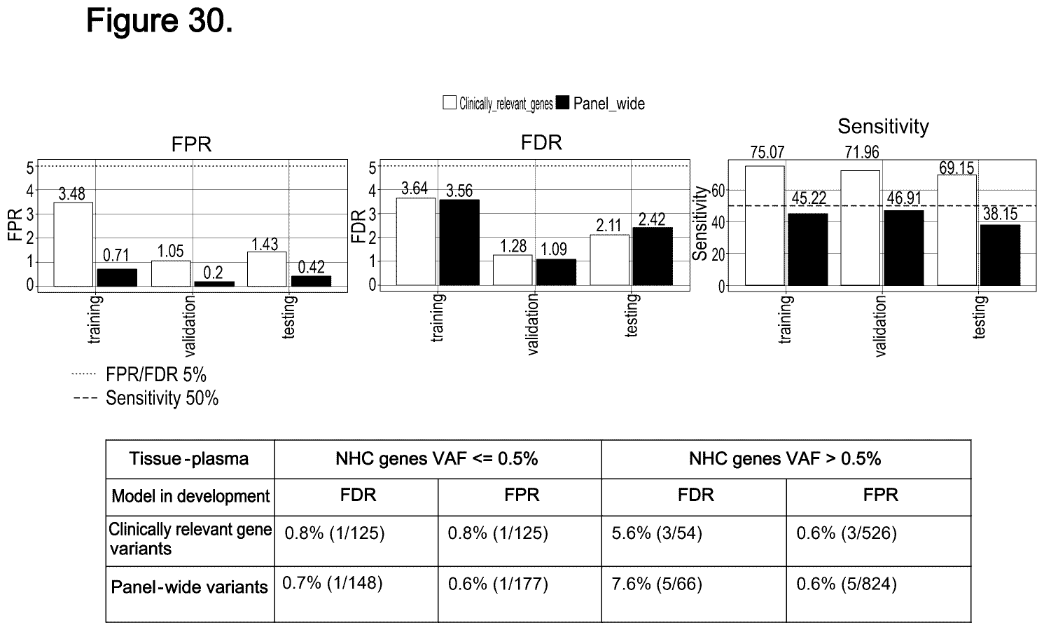

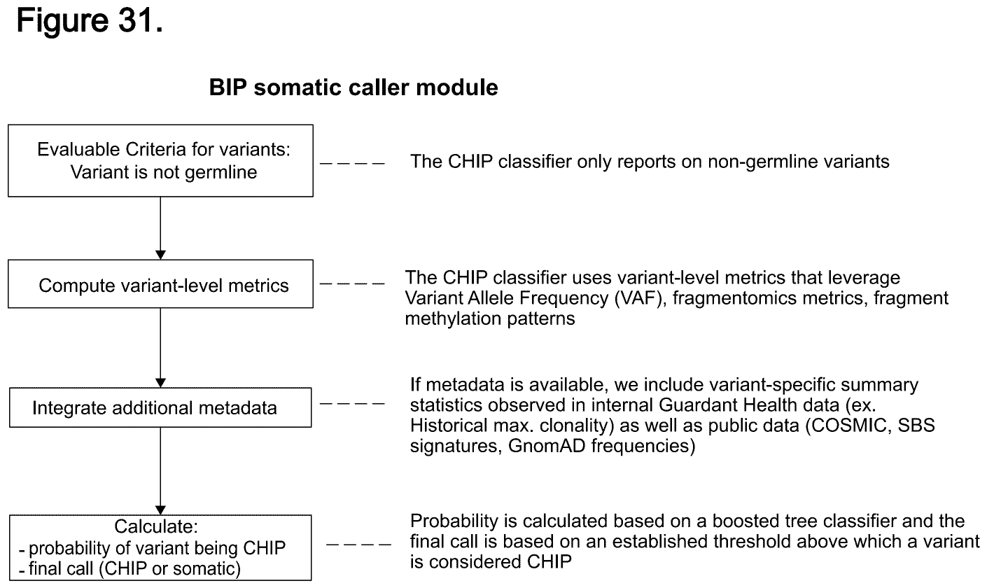

The described multi-feature model was then applied to Actionable variant performance: buffy-plasma matched samples. Current classifier achieved 100% accuracy and 0% FRP. As shown in FIG. 27, remarkably, the exemplified multi-feature classifier achieved 0% FRP. Further performance is shown in FIG. 28 when applied to actionable variant classification which indicates very low FPR, exemplary panel including approximately 80 genes. Actionable variant performance was assessed in plasma ctDNA—as shown in FIG. 29, with most CHIP calls in known KRAS CHIP variant K117N. Summary of current model performance is shown in FIG. 30. In testing data sensitivity went from 47% to 69% and FPR from 2.9% to 1.4% when compared to limited feature models. Deployment in a CHIP classifier algorithm as shown in FIG. 31.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

1. A method comprising:
determining sequence data of a plurality of sequence fragments associated with a plurality of genomic regions, wherein the sequence data comprises a plurality of sequence reads, wherein the plurality of sequence reads are sequenced from the plurality of sequence fragments from a plurality of samples, wherein each sample of the plurality of samples is labeled as a tumor derived or a non-tumor derived;
determining epigenetic data associated with the plurality of sequence fragments;
determining, based on the sequence data and epigenetic data, a plurality of features for a predictive model;
generating, based on the sequence data and epigenetic data, a predictive model according to the plurality of features.

2. The method of claim 1, wherein determining sequence data comprises obtaining a plurality of samples from a plurality of subjects, wherein the plurality of samples comprise a plurality of cell-free nucleic acids.

3. The method of claim 1, wherein the plurality of features comprise at least one of: fragment length, a variant VAF, variant CHIP to somatic ratio, APOBEC-related cancer marker, variant measurement variability, variant maximum clonality, an age related marker, variant clonality variance, population allele frequency, ratio of methylated to unmethylated fragments, a genomic region associated with a cancer type, a genomic region associated with a methylation status, a genomic region associated with hypomethylation, or a genomic region associated with therapy response.

4. The method of claim 3, wherein the fragment length is a mean length and/or length variance.

5. The method of claim 4, wherein the fragment length is a mononucleosome and/or dinucleosome associated.

6. The method of claim 4, wherein the age related marker is SBS88.

7. The method of claim 4, wherein the APOBEC=related cancer marker is SBS2.

8. The method of claim 4, wherein the variant measurement is one or more of:
variability, variant maximum clonality, and variant clonality variance

9. The method of claim 1, wherein the epigenetic data comprises at least one of: information regarding DNA methylation, histone states or modifications, inflammation-mediated cytosine damage products, or protein binding.

10. The method of claim 1, wherein determining the epigenetic data associated with the plurality of sequence fragments comprises determining a methylation state of the plurality of sequence fragments.

11. The method of claim 10, wherein determining the methylation state of the plurality of sequence fragments comprises determining at least one of: a methylation state vector or a methylated CpG density.

12. The method of claim 11, wherein determining the methylation state vector comprises:
aligning the plurality of sequence reads to a reference sequence;
determining, based on the aligning, a methylation status of one or more CpG sites in a sequence read of the plurality of sequence reads and a location of the one or more CpG sites; and
vectorizing the methylation status of the one or more CpG sites and the locations of the one or more CpG sites to generate the methylation state vector for the sequence read of the plurality of sequence reads.

13. The method of claim 11, wherein determining the methylated CpG density comprises:
aligning the plurality of sequence reads to a reference sequence;
determining, based on the aligning, a methylation status of one or more CpG sites in a sequence read of the plurality of sequence reads;
determining, based on the methylation status of the one or more CpG sites in the sequence read, that the sequence read is methylated or unmethylated;
determining, for the plurality of sequence reads, a count of methylated sequence reads and a count of unmethylated sequence reads; and
determining, based on the count of methylated sequence reads and the count of unmethylated sequence reads, the methylated CpG density.

14. The method of claim 1, wherein training the predictive model comprises application of a machine learning algorithm.

15. The method of claim 14, wherein the machine learning approach comprises at least one of: a discriminant analysis, a decision tree, a nearest neighbor (NN) algorithm, a Bayesian network, a clustering algorithm, a neural network, a support vector machine (SVM), a logistic regression algorithm, a linear regression algorithms, a Markov model, or a principal component analysis (PCA).

16. The method of claim 1, comprises retraining of the predictive model.

17. The method of claim 1, further comprising:
determining, for a subject, test sequence data comprising a plurality of sequence reads sequenced from a sample from the subject;
generating test epigenetic data and/or test fragmentomic data associated with the plurality of sequence fragments;
providing, to the predictive model, test sequence data, test epigenetic data, and test fragmentomic data of the subject; and
determining, based on the test sequence data, the test epigenetic data, and the test fragmentomic data of the subject, an origin of at least on sequence fragment in the sequence data.

18. The method of claim 1, comprising determining origin of at least on sequence fragment in the sequence data.

19. The method of claim 1, wherein the origin is one of tumor derived or non-tumor derived.

20. The method of claim 1, further comprising administering one or more therapies to the subject based on the origin being tumor derived.

21. The method of claim 1, wherein the therapies comprise administering chemotherapy, administering radiation therapy, or performing surgery to resect all or a portion of a tumor.

22-67. (canceled)
